(19) United States
(12) Patent Application Publication
(10) Pub. No.: US 2012/0010148 A1
(43) Pub. Date: Jan. 12, 2012

(54) COMBINATION THERAPIES USING NAP

(75) Inventors: Illana Gozes, Ramat Hasharon (IL); Gordon McCauley, Vancouver (CA)

(73) Assignees: ALLON THERAPEUTICS INC., Vancouver (CA); Ramot at Tel Aviv University Ltd., Tel Aviv (IL)

(21) Appl. No.: 13/174,167

(22) Filed: Jun. 30, 2011

Related U.S. Application Data
(63) Continuation-in-part of application No. PCT/CA09/01906, filed on Dec. 30, 2009.
(60) Provisional application No. 61/141,588, filed on Dec. 30, 2008.

(51) Int. Cl. A61K 38/16; A61P 25/18; A61P 25/00; A61K 38/08; A61P 25/28

(52) U.S. Cl. 514/17.8; 514/17.7; 514/17.9

(57) ABSTRACT

This invention relates to treatment of neurodegeneration, multiple sclerosis, or schizophrenia using an ADNF III polypeptide in combination with another therapeutic agent. Neurodegeneration, including neurodegeneration caused by dementia-related conditions, such as tauopathies, including Alzheimer's disease, and aging-related dementia, is treated by a combination of an ADNF III polypeptide and an acetylcholinesterase inhibitor. Multiple sclerosis is treated by a combination of an ADNF III polypeptide and glatiramer acetate. Schizophrenia is treated with a combination of an ADNF III peptide and an antipsychotic drug, selected from Aripiprazole, Clozapine, Ziprasidone, Resperidone, Quetiapine, and Olanzapine.

### CROSS-REFERENCE TO RELATED APPLICATIONS

This application is a continuation-in-part of PCT/CA2009/001906, filed Dec. 30, 2009, which claims priority to U.S. Provisional Patent Application No. 61/141,588, filed Dec. 30, 2008, contents of each of the above are hereby incorporated by reference in the entirety for all purposes.

### FIELD OF THE INVENTION

This invention relates to treatment of neurodegeneration, multiple sclerosis, or schizophrenia using an ADNF III polypeptide in combination with another therapeutic agent. Neurodegeneration, including neurodegeneration caused by dementia-related conditions, such as Alzheimer's disease, and aging-related dementia, is treated by a combination of an ADNF III polypeptide and an acetylcholinesterase inhibitor. Neurodegenration caused by dementia related conditions that is associated with non-Alzheimer's disease or aging-related tauopthy, for example, progressive supranuclear palsy treated with inhibitors of glycogen synthetase 3 beta, such as 4-Benzyl-2-(A-Naphtyl)-1,2,4-Thiadiazolidine-3,5-Dione, or other medications including but not limited to rasagiline. Multiple sclerosis is treated by a combination of an ADNF III polypeptide and glatiramer acetate or beta interferon. Schizophrenia is treated with a combination of an ADNF III peptide and an antipsychotic drug, selected from Aripiprazole, Clozapine, Ziprasidone, Resperidone, Quetiapine, and Olanzapine.

### BACKGROUND OF THE INVENTION

NAP, an 8-amino acid peptide (NAPVSIPQ=Asn-Ala-Pro-Val-Ser-Ile-Pro-Gln, SEQ ID NO:2), is derived from activity-dependent neuroprotective protein, ADNP or ADNF III (U.S. Pat. No. 6,613,740, Bassan et al., J. Neurochem. 72: 1283-1293 (1999)). The NAP sequence within the ADNF III gene is identical in rodents and humans (U.S. Pat. No. 6,613,740, Zamostiano, et al., J. Biol. Chem. 276:708-714 (2001); review Gozes, Pharmacol Ther. 114:146-154 (2007); and review Gozes et al., Curr Alzheimer Res. 6(5):455-460 (2009)).

In cell cultures, NAP has been shown to have neuroprotective activity at femtomolar concentrations against a wide variety of toxins (Bassan et al., 1999; Offen et al., Brain Res. 854:257-262 (2000)). In animal models simulating parts of the Alzheimer's disease pathology, NAP was protective as well (Bassan et al., 1999; Gozes et al., J. Pharmacol. Exp. Ther. 293:1091-1098 (2000); see also U.S. Pat. No. 6,613,740). In normal aging rats, intranasal administration of NAP improved performance in the Morris water maze. (Gozes et al., J. Mol. Neurosci. 19:175-178 (2002). Furthermore, NAP reduced infarct volume and motor function deficits after ischemic injury, by decreasing apoptosis (Leker et al., Stroke 33:1085-1092 (2002)) and reduced damage caused by closed head injury in mice by decreasing inflammation (Beni Adani et al., J. Pharmacol. Exp. Ther. 296:57-63 (2001); Romano et al., J. Mol. Neurosci. 18:37-45 (2002); Zaltzman et al., NeuroReport 14:481-484 (2003)). In a model of fetal alcohol syndrome, fetal death after intraperitoneal injection of alcohol was inhibited by NAP treatment (Spong et al., J. Pharmacol. Exp. Ther. 297:774-779 (2001); see also WO 00/53217). Utilizing radiolabeled peptides these studies showed that NAP can cross the blood-brain barrier and can be detected in rodents' brains either after intranasal treatment (Gozes et al., 2000) or intravenous injection (Leker et al., 2002) or intraperitoneal administration (Spong et al., 2001). D-NAP is an all D-amino acid derivative of NAP that is stable and orally available (Brenneman, et al., J Pharmacol Exp Ther. 309:1190-7 (2004)) and surprisingly exhibits similar biological activity (potency and efficacy) to NAP in the systems tested (WO0112654).

SAL, a 9-amino acid peptide (SALLRSIPA=Ser-Ala-Leu-Leu-Arg-Ser-Ile-Pro-Ala, SEQ ID NO:1), also known as ADNF-9 or ADNF-1, was identified as the shortest active form of ADNF (see U.S. Pat. No. 6,174,862). SAL has been shown in in vitro assays and in vivo disease models to keep neurons of the central nervous system alive in response to various insults (e.g., Gozes et al., 2000; Brenneman et al. (1998) J. Pharmacol. Exp. Ther. 285:619-627). D-SAL is an all D-amino acid derivative of SAL that is stable and orally available (Brenneman, et al., J Pharmacol Exp Ther. 309:1190-7 (2004)) and surprisingly exhibits similar biological activity (potency and efficacy) to SAL in the systems tested. Additional active peptides from ADNF-1 complexes are described in WO03/022226.

### BRIEF DESCRIPTION OF THE DRAWINGS

FIG. 1: Clozapine increases cell mortality and combination with NAP provides protection. Incubation with clozapine resulted in about 60% cell death, which was reversed by NAP treatment (10⁻¹⁵M).

FIG. 2: NAP does not affect cell mortality. Combination use of NAP and clozapine suppresses unwanted side effects associated with clozapine. As clozapine is widely used and represents a class of neuroleptics, this combination use has significant implications. Similar benefits are expected from combination use of drugs belonging to the clozapine class with NAP and related compounds.

FIG. 3: Serum deprivation results in PC12 cell death at different time points. The results show increased death after incubation for 48 hours, which was chosen as the optimal incubation time.

FIG. 4: Calibration of the Galantamine (Galanthamine) concentrations. Maximal survival is obtained with 0.1 mM concentration of galantamine. In additional experiments, there seems to be an inhibition of activity with increased concentration of galantamine at doses >0.1 mM (a bell-shape dose-dependent curve). The comparison is to 100% serum-deprived culture.

FIG. 5: Calibration of the NAP concentrations. NAP provided protection against serum deprivation as previously reported (Lagreze et al., Invest Ophthalrnol Vis Sci. 46(3):933-938 (2005)).

FIG. 6: Synergistic effect of NAP and galantamine (GAL): Experiments were conducted as above and NAP+galantamine show here an synergistic effect. The mixture of NAP+Galantamine (Gal) is significantly different from either alone at 0.1 mM Galantamine (GAL) and significantly different from NAP also at 0.05 mM galantamine (P<0.005). Again, the comparison is against serum deprived cultures (100%).

FIG. 7: Neither NAP nor galantamine protected against extensive cell death, however, their combination provided such protection. When comparing again in the absence and presence of serum and under conditions of >40% cell death, only the mixture provided protection (comparison are made against cultures supplemented with serum (Pv=P value).

FIG. 8: Validation of the experimental model. In order to validate the model for schizophrenic-like behavior, hyperactivity of the mice was examined in open field test for 5 consecutive sessions, each constituting of a 3-minute period making up for a total 15 minutes of testing per animal. The results are shown as average of the path in cm per animal in the three minute time period. ADNP+/+(Balbc—DD) represent the vehicle treated controls (n=11) exhibiting a significant difference from their vehicle (DD)-treated STOP+/−(heterozygous) littermates, which in turn demonstrated hyper locomotion behavior in an open field test (##P<0.01; Student's ttest), (Panel A). Further analysis showed that daily intranasal NAP administration significantly decreased the hyper-locomotion of the STOP heterozygous (STOP+/−) male mice (n=12; *** P<0.001; Student's t-test, STOP heterozygous DD vs. NAP treated STOP heterozygous mice) (Panel A). A trend for NAP activity was also observed in the control mice.

To test the hypothesis and justify posthoc pairwise comparisons, a genotype by treatment interaction was also implemented using a two-way ANOVA as outlined in the results section.

In order to evaluate the predictive validity of the model, the effect of clozapine (a known antipsychotic drug) on hyperactivity of the mice was examined in open field test as above. Male mice were daily treated for five weeks with clozapine (CLZ) or saline (IP). Results showed that even with a small sample size per group (3-4 STOP+/− mice) clozapine treatment significantly decreased the hyper-locomotion of the STOP heterozygous treated mice (** P<0.01) (Panel B).

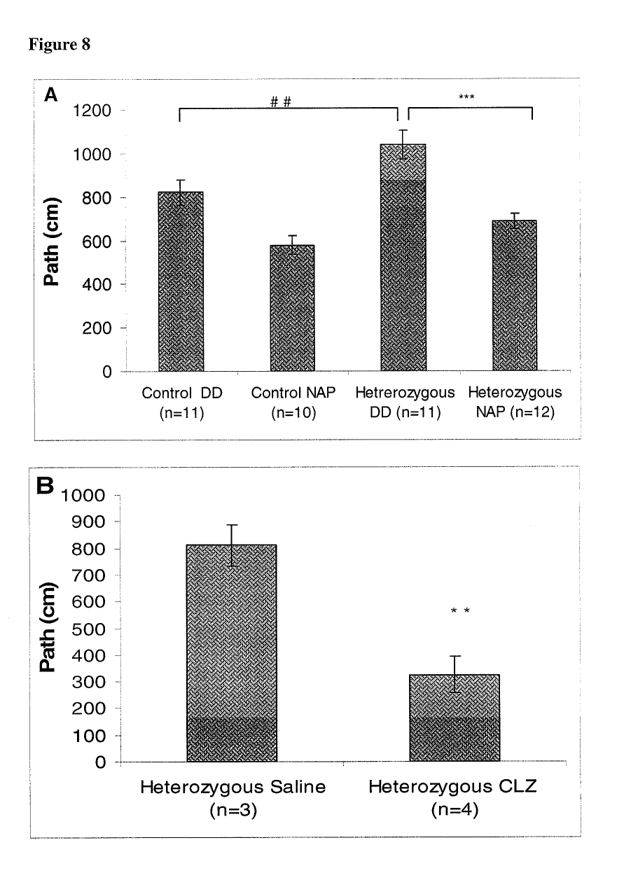

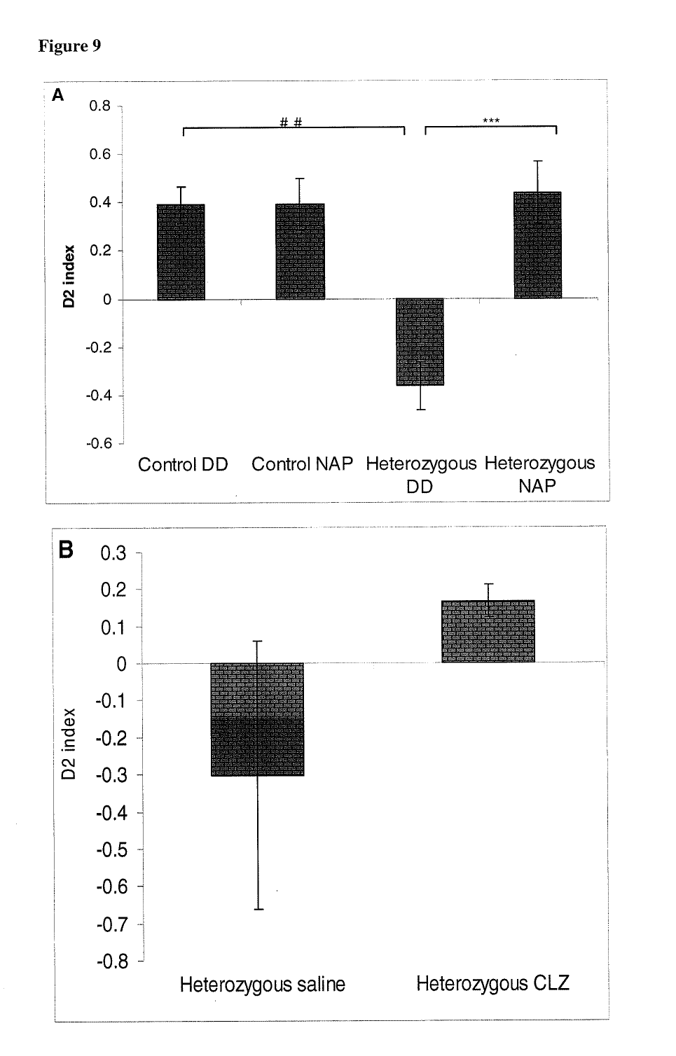

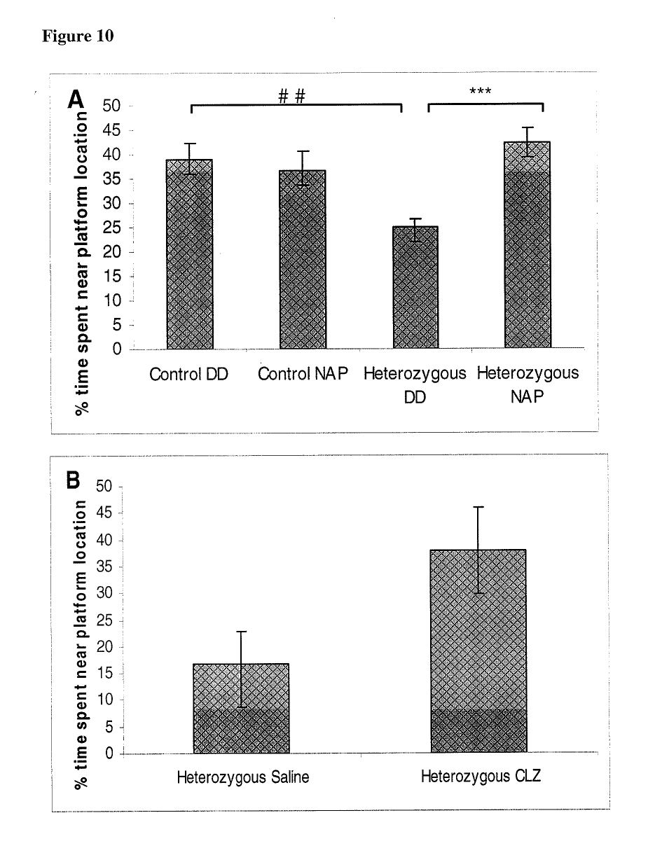

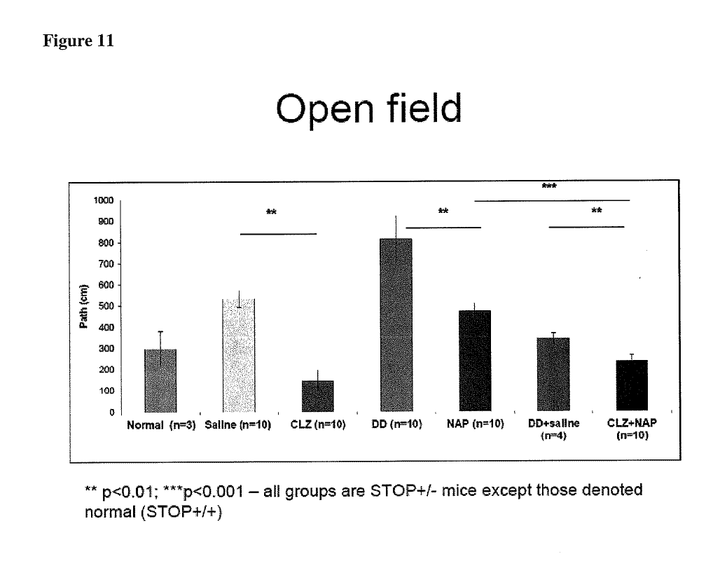

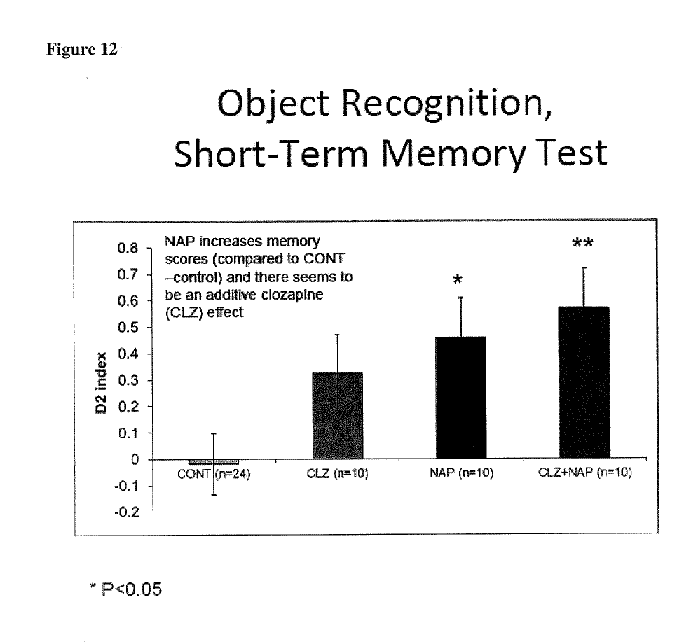

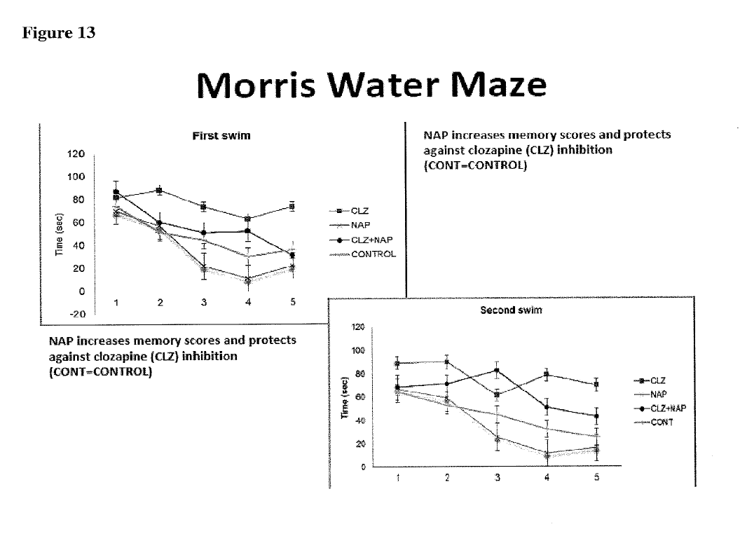

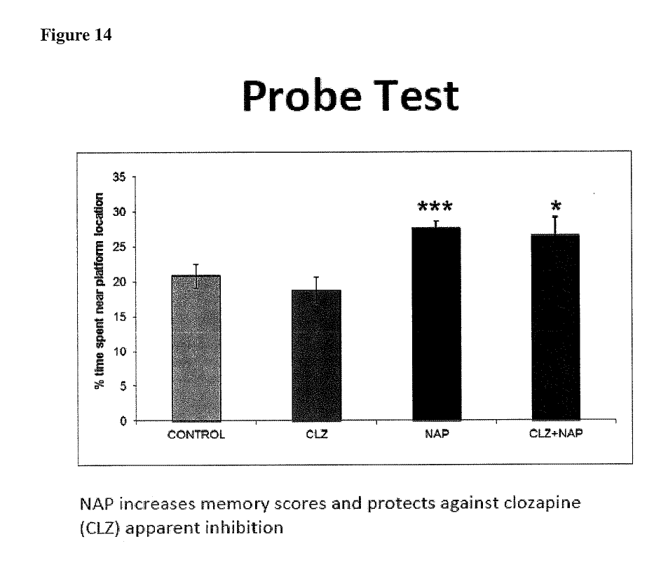

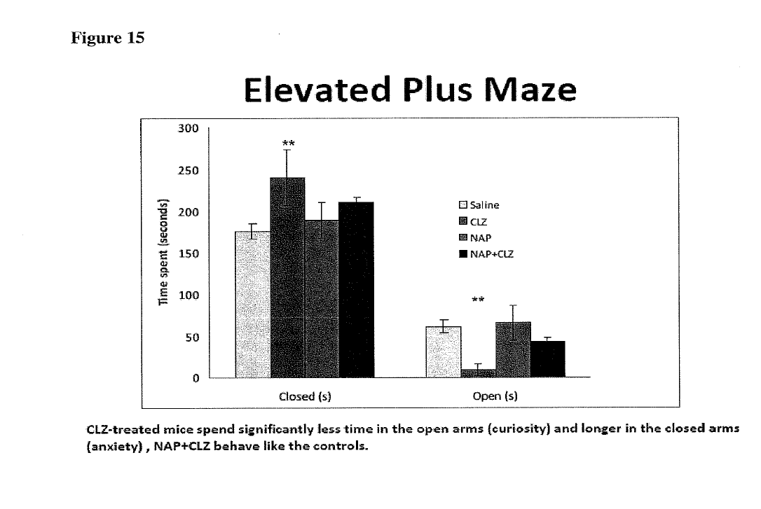

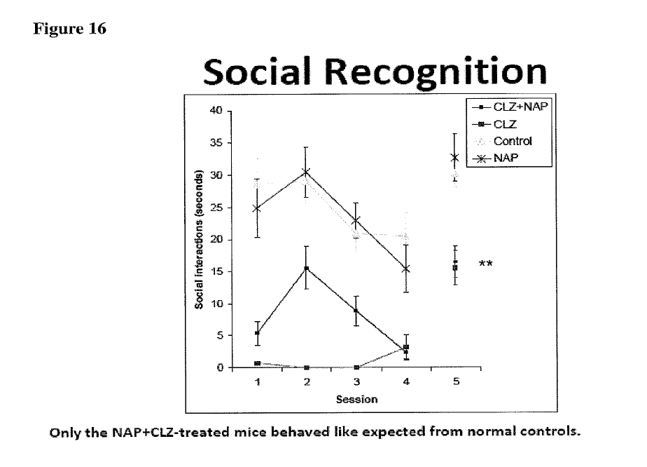

FIG. 9: NAP treatment increases cognitive function in STOP heterozygous mice in the object recognition and discrimination test. The same experimental groups described in FIG. 8 were further subjected to object recognition and discrimination test as follows. 3 h after a first session (performed with two identical objects, which showed no differences between the tested groups), the vehicle (DD)-treated STOP+/− mice showed significant deficits in recognizing the novel object and seemed to significantly prefer the familiar object (##P<0.01; Student's t-test). NAP treatment ameliorated completely the cognitive impairment associated with STOPdeficiency in the STOP+/− mice (***P<0.001; Student's t-test), (Panel A). To test the hypothesis and justify posthoc pairwise comparisons, a genotype by treatment interaction was also implemented using a two-way ANOVA as outlined in the results section. Clozapine treatment showed only a trend for improvement (Panel B).

FIG. 10: NAP treatment of STOP+/− ameliorates spatial memory deficits. The same experimental groups described in FIG. 8 were used for the Moris water maze test. The results of the probe test are depicted. The percentage of time (out of 90 seconds total exploration time) spent in the area of the water maze where the platform used to be (on the 5th day of training) was calculated. This measure is indicative of spatial memory. Results showed a significant difference between normal mice and STOP heterozygous mice (STOP+/−) [##P<0.01; Balbc (STOP+/+treated with vehicle-DD) compared with STOP+/−(heterozygous) littermates treated with vehicle-DD). NAP treatment significantly improved the performance of the STOP+/− mice (*** P<0.001; STOP+/−treated with DD compared to STOP+/−, treated with NAP). Furthermore, the values obtained for the NAP-treated STOP+/−mice (heterozygous) were similar to the values obtained for the control (STOP+/+normal mice, suggesting complete amelioration of the STOP-deficiency-associated deficit (Panel A). To test the hypothesis and justify posthoc pairwise comparisons, a genotype by treatment interaction was also implemented using a two-way ANOVA as outlined in the results section.

The differences among heterozygous mice treated with CLZ and heterozygous mice treated with saline showed trend that did not reach statistical significance although a trend was observed.

FIG. 11: Results of an open field test. Mice receiving CLZ treatment showed reduced activity, whereas NAP+CLZ treatment produced further reduction of activity than NAP treatment alone.

FIG. 12: Results of an object recognition test. Mice receiving NAP+CLZ treatment had the most increase in memory compared to both NAP treatment and CLZ treatment alone.

FIG. 13: Results of Morris Water Maze test. In the NAP+CLZ combination treatment group NAP showed protection against CLZ memory inhibition.

FIG. 14: Results of a probe test. Mice receiving NAP+CLZ treatment appared to be protected by NAP against CLZ inhibition of memory.

FIG. 15: Results of an elevated plus maze test. CLZ treatment alone led to reduced curiosity and increased anxiety, whereas NAP reversed these effects as NAP+CLZ treated mice behaved similarly to normal controls.

FIG. 16: Results of a social recognition test. Again, NAP reversed the negative effects from CLZ treatment alone and NAP+CLZ treated mice behaved similarly to normal controls.

### BRIEF SUMMARY OF THE INVENTION

In the first aspect, the invention provides an ADNF combination therapy or method of treating or preventing neurodegeneration caused by a dementia-related disorder (such as a dementia caused by a tauopathy or the aging process) in a human subject. For example, the dementia-related disorder may be a dementia related to Alzheimer's disease. The combination therapy includes a step of administering to the human subject an ADNF III polypeptide with an active core site of Asn-Ala-Pro-Val-Ser-Ile-Pro-Gln (SEQ ID NO:2); and an acetylcholinesterase inhibitor. In some embodiments, the acetylcholinesterase inhibitor is selected from huperzine, Huprines, methanesulfonyl fluoridemetrifonate, physostigmine, neostigmine, pyridostigmine, ambenonium, demarcarium, rivastigmine, galantamine, donepezil, Tacrine, Edrophonium, Phenothiazine, 4-Benzyl-2-(A-Naphtyl)-1,2,4-Thiadiazolidine-3,5-Dione, and rasaginile (azilect). In the alternative, the combination therapy comprises administering to the human subject an ADNF III polypeptide with an active core site of Asn-Ala-Pro-Val-Ser-Ile-Pro-Gln (SEQ ID NO:2) and an inhibitor of tau protein aggregation, such as methylene blue (Rember), phenylthiazolyl-hydrazide (PTH), and aminothienopyridazines (ATPZs).

In one embodiment, the ADNF III polypeptide is a full length ADNF III polypeptide.

In one embodiment, the ADNF III polypeptide used in the ADNF combination therapy to treat a dementia-related disease has the formula (R¹)x-Asn-Ala-Pro-Val-Ser-Ile-Pro-Gln-(R²)y (SEQ ID NO:13) in which R¹ is an amino acid sequence comprising from 1 to about 40 amino acids wherein each amino acid is independently selected from the group consisting of naturally occurring amino acids and amino acid analogs; R² is an amino acid sequence comprising from 1 to about 40 amino acids wherein each amino acid is independently selected from the group consisting of naturally occurring amino acids and amino acid analogs; and x and y are independently selected and are equal to zero or one.

In another embodiment, the ADNF III polypeptide is Asn-Ala-Pro-Val-Ser-Ile-Pro-Gln (SEQ ID NO:2).

The ADNF polypeptides can include D-amino acids. In a preferred embodiment the D-amino acid is with the active core site sequence disclosed above. In a further preferred embodiment, the active core site of the ADNF III polypeptide comprises all D-amino acids.

Exemplary ADNF III polypeptides for the ADNF combination therapy to treat dementia-related disorders include Gly-Gly-Asn-Ala-Pro-Val-Ser-Ile-Pro-Gln (SEQ ID NO:9); Leu-Gly-Gly-Asn-Ala-Pro-Val-Ser-Ile-Pro-Gln-Gln-Ser (SEQ ID NO:10); Leu-Gly-Leu-Gly-Gly-Asn-Ala-Pro-Val-Ser-Ile-Pro-Gln-Gln-Ser (SEQ ID NO:11); Ser-Val-Arg-Leu-Gly-Leu-Gly-Gly-Asn-Ala-Pro-Val-Ser-Ile-Pro-Gln-Gln-Ser (SEQ ID NO:12); and Asn-Ala-Pro-Val-Ser-Ile-Pro-Gln (SEQ ID NO:2).

In another embodiment, the ADNF III polypeptide has up to about 20 amino acids at one or both of the N-terminus and the C-terminus of the active core site. In further embodiments, ADNF III polypeptide contains a covalently bound lipophilic moiety to enhance penetration or activity.

The ADNF combination therapy to treat dementia related disorders, e.g., Alzheimer's disease and age-related dementia, can also be performed using an ADNF I peptide in place of the ADNF III peptides listed above. The core active site of the ADNF I peptide has an amino acid sequence of SEQ ID NO:1. In some embodiments, the ADNF I peptide comprise the ADNF I core active site sequence. Examples of such peptides include a full-length ADNF I protein, e.g., a full-length human ADNF I protein; and SEQ ID NOs:3-8. The polypeptide comprising an ADNF I core active site can include D-amino acid residues. In some embodiments, the D-amino acid residues are found in the ADNF I core active site sequence and in one embodiment, all of the ADNF I core active amino acid residues are D-amino acids. In another preferred embodiment, the ADNF peptide is the ADNF I core active site peptide, e.g., SEQ ID NO:1. The ADNF I core active site peptide can include one or more D-amino acid residues. In a further preferred embodiment, the ADNF I core active site peptide consists of all D-amino acid residues, i.e., SEQ ID NO:1 is all D-amino acids.

In some embodiments, the patient receiving the combination treatment described above suffers from a tauopathy such as Alzheimer's disease, Parkinson's disease, frontotemporal dementia (FTD), corticobasal degeneration, frontotemporal lobar degeneration (Pick's disease), progressive supranuclear palsy (PSP), and amyotrophic lateral sclerosis (ALS, or Lou Gehrig's Disease).

In the second aspect, the invention provides an ADNF combination therapy or method of treating or preventing multiple sclerosis (MS) in a human subject. The combination therapy includes a step of administering to the human subject an ADNF III polypeptide with an active core site of Asn-Ala-Pro-Val-Ser-Ile-Pro-Gln (SEQ ID NO:2); and glatiramer acetate or beta interferon.

In one embodiment, the ADNF III polypeptide used in the MS combination therapy is a full length ADNF III polypeptide.

In one embodiment, the ADNF III polypeptide used in the MS combination therapy has the formula (R¹)x-Asn-Ala-Pro-Val-Ser-Ile-Pro-Gln-(R²)y (SEQ ID NO:13) in which R¹ is an amino acid sequence comprising from 1 to about 40 amino acids wherein each amino acid is independently selected from the group consisting of naturally occurring amino acids and amino acid analogs; R² is an amino acid sequence comprising from 1 to about 40 amino acids wherein each amino acid is independently selected from the group consisting of naturally occurring amino acids and amino acid analogs; and x and y are independently selected and are equal to zero or one.

In another embodiment, the ADNF III polypeptide used in the MS combination therapy is Asn-Ala-Pro-Val-Ser-Ile-Pro-Gln (SEQ ID NO:2).

The ADNF III polypeptides can include D-amino acids. In a preferred embodiment the D-amino acid is with the active core site sequence disclosed above. In a further preferred embodiment, the active core site of the ADNF III polypeptide comprises all D-amino acids.

Exemplary ADNF III polypeptides used in the MS combination therapy include Gly-Gly-Asn-Ala-Pro-Val-Ser-Ile-Pro-Gln (SEQ ID NO:9); Leu-Gly-Gly-Asn-Ala-Pro-Val-Ser-Ile-Pro-Gln-Gln-Ser (SEQ ID NO:10); Leu-Gly-Leu-Gly-Gly-Asn-Ala-Pro-Val-Ser-Ile-Pro-Gln-Gln-Ser (SEQ ID NO:11); Ser-Val-Arg-Leu-Gly-Leu-Gly-Gly-Asn-Ala-Pro-Val-Ser-Ile-Pro-Gln-Gln-Ser (SEQ ID NO:12); and Asn-Ala-Pro-Val-Ser-Ile-Pro-Gln (SEQ ID NO:2).

In another embodiment, the ADNF III polypeptide used in the MS combination therapy has up to about 20 amino acids at one or both of the N-terminus and the C-terminus of the active core site. In further embodiments, ADNF III polypeptide contains a covalently bound lipophilic moiety to enhance penetration or activity.

The ADNF MS combination therapy can also be performed using an ADNF I peptide in place of the ADNF III peptides listed above. The core active site of the ADNF I peptide has an amino acid sequence of SEQ ID NO:1. In some embodiments, the ADNF I peptide comprise the ADNF I core active site sequence. Examples of such peptides include a full-length ADNF I protein, e.g., a full-length human ADNF I protein; and SEQ ID NOs:3-8. The polypeptide comprising an ADNF I core active site can include D-amino acid residues. In some embodiments, the D-amino acid residues are found in the ADNF I core active site sequence and in one embodiment, all of the ADNF I core active amino acid residues are D-amino acids. In another preferred embodiment, the ADNF peptide is the ADNF I core active site peptide, e.g., SEQ ID NO:1. The ADNF I core active site peptide can include one or more D-amino acid residues. In a further preferred embodiment, the ADNF I core active site peptide consists of all D-amino acid residues, i.e., SEQ ID NO:1 is all D-amino acids.

In some embodiments, the ADNF polypeptide comprising or consisting of the core active sequence (SEQ ID NO:1 or 2) is used in combination with interferon β-1b (also known as Betaferon or Betaseron) to treat a patient suffering from MS.

In the third aspect, the invention provides an ADNF combination therapy or method of treating or preventing schizophrenia in a human subject. The combination therapy includes a step of administering to the human subject an ADNF III polypeptide with an active core site of Asn-Ala-Pro-Val-Ser-Ile-Pro-Gln (SEQ ID NO:2); and an anti-psychotic agent. In some embodiments, the anti-psychotic agent is selected from Aripiprazole, Clozapine, Ziprasidone, Resperidone, Quetiapine, and Olanzapine.

In one embodiment, the ADNF III polypeptide used in the schizophrenia combination therapy is a full length ADNF III polypeptide.

In one embodiment, the ADNF III polypeptide used in the schizophrenia combination therapy has the formula (R¹)x-Asn-Ala-Pro-Val-Ser-Ile-Pro-Gln-(R²)y (SEQ ID NO:13) in which R¹ is an amino acid sequence comprising from 1 to about 40 amino acids wherein each amino acid is independently selected from the group consisting of naturally occurring amino acids and amino acid analogs; R² is an amino acid sequence comprising from 1 to about 40 amino acids wherein each amino acid is independently selected from the group consisting of naturally occurring amino acids and amino acid analogs; and x and y are independently selected and are equal to zero or one.

In another embodiment, the ADNF III polypeptide used in the schizophrenia combination therapy is Asn-Ala-Pro-Val-Ser-Ile-Pro-Gln (SEQ ID NO:2).

The ADNF III polypeptides can include D-amino acids. In a preferred embodiment the D-amino acid is with the active core site sequence disclosed above. In a further preferred embodiment, the active core site of the ADNF III polypeptide comprises all D-amino acids.

Exemplary ADNF III polypeptides used in the schizophrenia combination therapy include Gly-Gly-Asn-Ala-Pro-Val-Ser-Ile-Pro-Gln (SEQ ID NO:9); Leu-Gly-Gly-Asn-Ala-Pro-Val-Ser-Ile-Pro-Gln-Gln-Ser (SEQ ID NO:10); Leu-Gly-Leu-Gly-Gly-Asn-Ala-Pro-Val-Ser-Ile-Pro-Gln-Gln-Ser (SEQ ID NO:11); Ser-Val-Arg-Leu-Gly-Leu-Gly-Gly-Asn-Ala-Pro-Val-Ser-Ile-Pro-Gln-Gln-Ser (SEQ ID NO:12); and Asn-Ala-Pro-Val-Ser-Ile-Pro-Gln (SEQ ID NO:2).

In another embodiment, the ADNF III polypeptide used in the schizophrenia combination therapy has up to about 20 amino acids at one or both of the N-terminus and the C-terminus of the active core site. In further embodiments, ADNF III polypeptide contains a covalently bound lipophilic moiety to enhance penetration or activity.

The ADNF schizophrenia combination therapy can also be performed using an ADNF I peptide in place of the ADNF III peptides listed above. The core active site of the ADNF I peptide has an amino acid sequence of SEQ ID NO:1. In some embodiments, the ADNF I peptide comprise the ADNF I core active site sequence. Examples of such peptides include a full-length ADNF I protein, e.g., a full-length human ADNF I protein; and SEQ ID NOs:3-8. The polypeptide comprising an ADNF I core active site can include D-amino acid residues. In some embodiments, the D-amino acid residues are found in the ADNF I core active site sequence and in one embodiment, all of the ADNF I core active amino acid residues are D-amino acids. In another preferred embodiment, the ADNF peptide is the ADNF I core active site peptide, e.g., SEQ ID NO:1. The ADNF I core active site peptide can include one or more D-amino acid residues. In a further preferred embodiment, the ADNF I core active site peptide consists of all D-amino acid residues, i.e., SEQ ID NO:1 is all D-amino acids.

In one particular embodiment, the ADNF III peptide used in combination treatement for schizophrenia is NAP (having the amino acid sequence set forth in SEQ ID NO:2), and the anti-psychotic agent is Clozapine or Olanzapine. As shown in the examples herein, the ADNF III polypeptide (e.g., NAP) are useful for reducing, ameliorating, or reversing the undesirable side effects of Clozapine treatment, such as suppressed learning and memory capacity, increased anxiety, and altered social behavior (impaired social recognition), thereby providing added benefits to those receiving Clozapine treatment. While Clozapine is an antipsychotic medication approved for used in the treatment of schizophrenia, it is also used off-label for treating psychosis in L-Dopa-treated patients; treating psychotic symptoms occurring in patients with dementia of the Lewy-body-type; treating otherwise resistant bipolar disorder, especially acute episodes of mania; treating intractable chronic insomnia (especially if all other measures have failed); treating schizoid personality disorder; treating otherwise resistant paranoid personality disorder and delusional disorder; treating essential tremor (second line to propranolol and primidone). Various structurally and/or functionally similar antipsychotic drugs are known for similar therapeutic uses, they include first-generation antipsychotics (or typical antipsychotics, i.e., chlorpromazine, fluphenazine, haloperidol, loxapine, perphenazine, thiothixene, thioridazine, or trifluoperazine) and second-generation antipsychotics (or atypical antipsychotics, i.e., aripiprazole, clozapine, olanzapine, quetiapine, risperidone, ziprasidone, dibenzodiazepene, paliperidone, asenapine, or iloperidone). Due to their similarities to Clozapine, especially those with distinct structural similarities to Clozapine, such as loxapine, these therapeutic agents can also have their undesirable side effects reduced, ameliorated, or reversed during their therapeutic use by co-administration with the ADNF III peptide described herein (e.g., NAP).

In some cases, NAP is administered separately, e.g., by intranasal delivery or systemic administation, from the anti-psychotic agent, e.g., administration of Clozapine by injection or oral formualtion. In other cases, the ADNF III peptide (e.g., NAP) may be administered with the anti-psychotic agent (e.g., Clozapine) in one single formulation. As such, a composition comprising an effective amount of the ADNF III peptide (e.g., NAP) and an effective amount of an anti-psychotic agent (e.g., Clozapine), optionally with a pharmaceutically acceptable diluent, carrier or excipient, is another aspect of the present invention.

### DEFINITIONS

The phrase “ADNF polypeptide” refers to one or more activity dependent neurotrophic factors (ADNF) that have an active core site comprising the amino acid sequence of SALLRSIPA (referred to as “SAL” or “ADNF-9,” SEQ ID NO:1) or NAPVSIPQ (referred to as “NAP,” SEQ ID NO:2), or conservatively modified variants thereof that have neurotrophic/neuroprotective activity as measured with in vitro cortical neuron culture assays described by, e.g., Hill et al., Brain Res. 603:222-233 (1993); Brenneman & Gozes, J. Clin. Invest. 97:2299-2307 (1996), Forsythe & Westbrook, J. Physiol. Lond. 396:515 (1988). An ADNF polypeptide can be an ADNF I polypeptide, an ADNF III polypeptide, their alleles, polymorphic variants, analogs, interspecies homolog, any subsequences thereof (e.g., SALLRSIPA (SEQ ID NO:1) or NAPVSIPQ (SEQ ID NO:2)) or lipophilic variants that exhibit neuroprotective/neurotrophic action on, e.g., neurons originating in the central nervous system either in vitro or in vivo. An “ADNF polypeptide” can also refer to a mixture of an ADNF I polypeptide and an ADNF III polypeptide.

The term “ADNF I” refers to an activity dependent neurotrophic factor polypeptide having a molecular weight of about 14,000 Daltons with a pI of 8.3±0.25. As described above, ADNF I polypeptides have an active site comprising an amino acid sequence of Ser-Ala-Leu-Leu-Arg-Ser-Ile-Pro-Ala (also referred to as “SALLRSIPA” or “SAL” or “ADNF-9,” SEQ ID NO:1). See Brenneman & Gozes, J. Clin. Invest. 97:2299-2307 (1996), Glazner et al., Anat. Embryol. ((Berl). 200:65-71 (1999), Brenneman et al., J. Pharm. Exp. Ther., 285:619-27 (1998), Gozes & Brenneman, J. Mol. Neurosci. 7:235-244 (1996), and Gozes et al., Dev. Brain Res. 99:167-175 (1997), all of which are herein incorporated by reference. Unless indicated as otherwise, “SAL” refers to a peptide having an amino acid sequence of Ser-Ala-Leu-Leu-Arg-Ser-Ile-Pro-Ala (SEQ ID NO:1), not a peptide having an amino acid sequence of Ser-Ala-Leu. A full length amino acid sequence of ADNF I can be found in WO 96/11948, herein incorporated by reference in its entirety.

The phrase “ADNF III polypeptide” or “ADNF III” also called activity-dependent neuroprotective protein (ADNP) refers to one or more activity dependent neurotrophic factors (ADNF) that have an active core site comprising the amino acid sequence of NAPVSIPQ (referred to as “NAP,” SEQ ID NO:2), or conservatively modified variants thereof that have neurotrophic/neuroprotective activity as measured with in vitro cortical neuron culture assays described by, e.g., Hill et al., Brain Res. 603, 222-233 (1993); Gozes et al., Proc. Natl. Acad. Sci. USA 93, 427-432 (1996). An ADNF polypeptide can be an ADNF III polypeptide, allelelic or polymorphic variant, analog, interspecies homolog, or any subsequences thereof (e.g., NAPVSIPQ, SEQ ID NO:2) that exhibit neuroprotective/neurotrophic action on, e.g., neurons originating in the central nervous system either in vitro or in vivo. ADNF III polypeptides can range from about eight amino acids and can have, e.g., between 8-20, 8-50, 10-100 or about 1000 or more amino acids.

Full length human ADNF III has a predicted molecular weight of 123,562.8 Da (>1000 amino acid residues) and a theoretical pI of about 6.97. As described above, ADNF III polypeptides have an active site comprising an amino acid sequence of Asn-Ala-Pro-Val-Ser-Ile-Pro-Gln (also referred to as “NAPVSIPQ” or “NAP,” SEQ ID NO:2). See Zamostiano et al., J. Biol. Chem. 276:708-714 (2001) and Bassan et al., J. Neurochem. 72:1283-1293 (1999), each of which is incorporated herein by reference. Unless indicated as otherwise, “NAP” refers to a peptide having an amino acid sequence of Asn-Ala-Pro-Val-Ser-Ile-Pro-Gln (SEQ ID NO:2), not a peptide having an amino acid sequence of Asn-Ala-Pro. Full-length amino acid and nucleic acid sequences of ADNF III can be found in WO 98/35042, WO 00/27875, U.S. Pat. Nos. 6,613,740 and 6,649,411. The Accession number for the human sequence is NP_{—}852107, see also Zamostiano et al., supra.

The term “subject” refers to any mammal, in particular human, at any stage of life. The term “contacting” is used herein interchangeably with the following: combined with, added to, mixed with, passed over, incubated with, flowed over, etc. Moreover, the ADNF III polypeptides or nucleic acids encoding them of the present invention can be “administered” by any conventional method such as, for example, parenteral, oral, topical, and inhalation and nasal routes. In some embodiments, parenteral and nasal application routes are employed.

The term “tauopathy” refers to a disease belonging to a class of neurodegenerative disorders caused by pathological aggregation of the tau protein in the so-called neurofibrillary tangles (NFT) in the human brain. Included in the general definition of tauopathies are Alzheimer's disease, Parkinson's disease, frontotemporal dementia, corticobasal degeneration, frontotemporal lobar degeneration (Pick's disease), and progressive supranuclear palsy (PSP).

A “mental disorder” or “mental illness” or “mental disease” or “psychiatric or neuropsychiatric disease or illness or disorder” refers to mood disorders (e.g., major depression, mania, and bipolar disorders), psychotic disorders (e.g., schizophrenia, schizoaffective disorder, schizophreniform disorder, delusional disorder, brief psychotic disorder, and shared psychotic disorder), personality disorders, anxiety disorders (e.g., obsessive-compulsive disorder and attention deficit disorders) as well as other mental disorders such as substance-related disorders, childhood disorders, dementia, autistic disorder, adjustment disorder, delirium, multi-infarct dementia, and Tourette's disorder as described in Diagnostic and Statistical Manual of Mental Disorders, Fourth Edition, (DSM IV). Typically, such disorders have a complex genetic and/or a biochemical component. As used herein, the term “schizophrenia” encompasses any mental/behavioral disorder generally fitting the diagnostic criteria set forth in DSM IV, including drug abuse-related behavioral disorders (e.g., schizophrenia-like behavior).

The terms “polypeptide,” “peptide,” and “protein” are used interchangeably herein to refer to a polymer of amino acid residues. Generally, a peptide refers to a short polypeptide. The terms apply to amino acid polymers in which one or more amino acid residue is an analog or mimetic of a corresponding naturally occurring amino acid, as well as to naturally occurring amino acid polymers.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. For the purposes of this application, amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. For the purposes of this application, amino acid mimetics refers to chemical compounds that have a structure that is different from the general chemical structure of an amino acid, but that functions in a manner similar to a naturally occurring amino acid.

Amino acids may include those having non-naturally occurring D-chirality, as disclosed in WO 01/12654, incorporated herein by reference, which may improve oral availability and other drug like characteristics of the compound. In such embodiments, one or more, and potentially all of the amino acids of NAP or the ADNF polypeptide will have D-chirality. The therapeutic use of peptides can be enhanced by using D-amino acids to provide longer half life and duration of action. However, many receptors exhibit a strong preference for L-amino acids, but examples of D-peptides have been reported that have equivalent activity to the naturally occurring L-peptides, for example, pore-forming antibiotic peptides, beta amyloid peptide (no change in toxicity), and endogenous ligands for the CXCR4 receptor. In this regard, NAP and ADNF polypeptides also retain activity in the D-amino acid form (Brenneman et al., J. Pharmacol. Exp. Ther. 309:1190-1197 (2004), see also WO0112654).

Amino acids may be referred to by either their commonly known three letter symbols or by the one-letter symbols recommended by the IUPAC-IUB Biochemical Nomenclature Commission. Nucleotides, likewise, may be referred to by their commonly accepted single-letter codes. The amino acids referred to herein are described by shorthand designations as follows:

TABLE I
Amino Acid Nomenclature

| Name | 3-letter | 1 letter |
|---|---|---|
| Alanine | Ala | A |
| Arginine | Arg | R |
| Asparagine | Asn | N |
| Aspartic Acid | Asp | D |
| Cysteine | Cys | C |
| Glutamic Acid | Glu | E |
| Glutamine | Gln | Q |
| Glycine | Gly | G |
| Histidine | His | H |
| Homoserine | Hse | — |
| Isoleucine | Ile | I |
| Leucine | Leu | L |
| Lysine | Lys | K |
| Methionine | Met | M |
| Methionine sulfoxide | Met (O) | — |
| Methionine methylsulfonium | Met (S—Me) | — |
| Norleucine | Nle | — |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following groups each contain amino acids that are conservative substitutions for one another:
1) Alanine (A), Glycine (G);
2) Serine (S), Threonine (T);
3) Aspartic acid (D), Glutamic acid (E);
4) Asparagine (N), Glutamine (Q);
5) Cysteine (C), Methionine (M);
6) Arginine (R), Lysine (K), Histidine (H);
7) Isoleucine (I), Leucine (L), Valine (V); and
8) Phenylalanine (F), Tyrosine (Y), Tryptophan (W). (see, e.g., Creighton, Proteins (1984)).

One of skill in the art will appreciate that many conservative variations of the nucleic acid and polypeptide sequences provided herein yield functionally identical products. For example, due to the degeneracy of the genetic code, “silent substitutions” (i.e., substitutions of a nucleic acid sequence that do not result in an alteration in an encoded polypeptide) are an implied feature of every nucleic acid sequence that encodes an amino acid. Similarly, “conservative amino acid substitutions,” in one or a few amino acids in an amino acid sequence are substituted with different amino acids with highly similar properties (see the definitions section, supra), are also readily identified as being highly similar to a disclosed amino acid sequence, or to a disclosed nucleic acid sequence that encodes an amino acid. Such conservatively substituted variations of each explicitly listed nucleic acid and amino acid sequences are a feature of the present invention.

The term “isolated,” “purified,” or “biologically pure” refers to a material that is substantially or essentially free from components that normally accompany it as found in its native state.

“An amount sufficient” or “an effective amount” or a “therapeutically effective amount” is that amount of a given NAP or ADNF polypeptide that exhibits the activity of interest or which provides either a subjective relief of a symptom(s) or an objectively identifiable improvement as noted by the clinician or other qualified observer. In therapeutic applications, the ADNF combination therapeutic of the invention is administered to a patient in an amount sufficient to reduce or eliminate symptoms of Alzheimer's disease, multiple sclerosis, or psychosis, e.g., schizophrenia. An amount adequate to accomplish this is defined as the “therapeutically effective dose.” The dosing range varies with the ADNF polypeptide and additional therapeutic used, the route of administration and the potency of the particular drugs, as further set out below, and in patents such as Canadian Patent No. 2,202,496,U.S. Pat. No. 6,174,862 and U.S. Pat. No. 6,613,740, herein incorporated by reference in their entirety.

“Inhibitors,” “activators,” and “modulators” of expression or of activity are used to refer to inhibitory, activating, or modulating molecules, respectively, identified using in vitro and in vivo assays for expression or activity, e.g., ligands, agonists, antagonists, and their homologs and mimetics. The term “modulator” includes inhibitors and activators. Inhibitors are agents that, e.g., inhibit expression of a polypeptide or polynucleotide of the invention or bind to, partially or totally block stimulation or enzymatic activity, decrease, prevent, delay activation, inactivate, desensitize, or down regulate the activity of a polypeptide or polynucleotide of the invention, e.g., antagonists. Activators are agents that, e.g., induce or activate the expression of a polypeptide or polynucleotide of the invention or bind to, stimulate, increase, open, activate, facilitate, enhance activation or enzymatic activity, sensitize or up regulate the activity of a polypeptide or polynucleotide of the invention, e.g., agonists. Modulators include naturally occurring and synthetic ligands, antagonists, agonists, small chemical molecules and the like. Assays to identify inhibitors and activators include, e.g., applying putative modulator compounds to cells, in the presence or absence of a polypeptide or polynucleotide of the invention and then determining the functional effects on a polypeptide or polynucleotide of the invention activity. Samples or assays comprising a polypeptide or polynucleotide of the invention that are treated with a potential activator, inhibitor, or modulator are compared to control samples without the inhibitor, activator, or modulator to examine the extent of effect. Control samples (untreated with modulators) are assigned a relative activity value of 100%. Inhibition is achieved when the activity value of a polypeptide or polynucleotide of the invention relative to the control is about 80%, optionally 50% or 25-1%. Activation is achieved when the activity value of a polypeptide or polynucleotide of the invention relative to the control is 110%, optionally 150%, optionally 200-500%, or 1000-3000% higher.

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, oligonucleotide, etc. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 Daltons and less than about 2500 Daltons, preferably less than about 2000 Daltons, preferably between about 100 to about 1000 Daltons, more preferably between about 200 to about 500 Daltons.

### DETAILED DESCRIPTION OF THE INVENTION

### I. Introduction

ADNF peptides and related compounds are combined with other therapeutics to provide synergistic reduction of symptoms of Alzheimer's disease, multiple sclerosis, or psychosis, including schizophrenia as well as less abudant tauopathies relative to Alzheimer's disease such as progressive supranuclear palsy. This improved treatment is referred to as “ADNF combination treatment.”

### II. ADNF Polypeptides

In one embodiment, the ADNF polypeptides of the present invention comprise the following amino acid sequence: (R¹)ₓ-Asn-Ala-Pro-Val-Ser-Ile-Pro-Gln-(R²)_{y} (SEQ ID NO:13) and conservatively modified variations thereof. In this designation, R¹ denotes the orientation of the amino terminal (NH₂ or N-terminal) end and R² represents the orientation of the carboxyl terminal (COOH or C-terminal) end.

In the above formula, R¹ is an amino acid sequence comprising from 1 to about 40 amino acids, wherein each amino acid is independently selected from the group consisting of naturally occurring amino acids and amino acid analogs. The term “independently selected” is used herein to indicate that the amino acids making up the amino acid sequence R¹ may be identical or different (e.g., all of the amino acids in the amino acid sequence may be threonine, etc.). Moreover, as previously explained, the amino acids making up the amino acid sequence R¹ may be either naturally occurring amino acids, or known analogues of natural amino acids that functions in a manner similar to the naturally occurring amino acids (i.e., amino acid mimetics and analogs). Suitable amino acids that can be used to form the amino acid sequence R¹ include, but are not limited to, those listed in Table I, infra. The indexes “x” and “y” are independently selected and can be equal to one or zero.

As with R¹, R², in the above formula, is an amino acid sequence comprising from 1 to about 40 amino acids, wherein each amino acid is independently selected from the group consisting of naturally occurring amino acids and amino acid analogs. Moreover, as with R¹, the amino acids making up the amino acid sequence R² may be identical or different, and may be either naturally occurring amino acids, or known analogues of natural amino acids that functions in a manner similar to the naturally occurring amino acids (i.e., amino acid mimetics and analogs). Suitable amino acids that can be used to form R² include, but are not limited to, those listed in Table I, infra.

As used herein, “NAP” or “NAP peptide” refers to the formula above where x and y both equal 0. “NAP related peptide” refers to any of the other variants of NAP which are described the formula.

R¹ and R² are independently selected. If R¹, R² are the same, they are identical in terms of both chain length and amino acid composition. For example, both R¹ and R² may be Val-Leu-Gly-Gly-Gly (SEQ ID NO:14). If R¹ and R² are different, they can differ from one another in terms of chain length and/or amino acid composition and/or order of amino acids in the amino acids sequences. For example, R¹ may be Val-Leu-Gly-Gly-Gly (SEQ ID NO:14), whereas R² may be Val-Leu-Gly-Gly (SEQ ID NO:15). Alternatively, R¹ may be Val-Leu-Gly-Gly-Gly (SEQ ID NO:14), whereas R² may be Val-Leu-Gly-Gly-Val (SEQ ID NO:16). Alternatives, R¹ may be Val-Leu-Gly-Gly-Gly (SEQ ID NO:14), whereas R² may be Gly-Val-Leu-Gly-Gly (SEQ ID NO:17).

Within the scope of the above formula, certain NAP and NAP related polypeptides are preferred, namely those in which x and y are both zero (i.e., NAP). Equally preferred are NAP and NAP related polypeptides in which x is one; R¹ Gly-Gly; and y is zero. Also equally preferred are NAP and NAP related polypeptides in which x is one; R¹ is Leu-Gly-Gly; y is one; and R² is -Gln-Ser. Also equally preferred are NAP and NAP related polypeptides in which x is one; R¹ is Leu-Gly-Leu-Gly-Gly- (SEQ ID NO:18); y is one; and R² is -Gln-Ser. Also equally preferred are NAP and NAP related polypeptides in which x is one; R¹ is Ser-Val-Arg-Leu-Gly-Leu-Gly-Gly-(SEQ ID NO:19); y is one; and R² is -Gln-Ser. Additional amino acids can be added to both the N-terminus and the C-terminus of the active peptide without loss of biological activity.

In another aspect, the present invention provides pharmaceutical compositions comprising one of the previously described NAP and NAP related polypeptides and an appropriate combination therapeutic in a pharmaceutically acceptable diluent, carrier or excipient, in an amount sufficient to reduce symptoms of Alzheimer's disease or other tauopathy including progressive supranuclear palsy, or MS, or psychosis, including schizophrenia. In one embodiment, the NAP or NAP related peptide has an amino acid sequence selected from the group consisting of SEQ ID NOs:2 and 9-12, and conservatively modified variations thereof.

In another embodiment, the ADNF polypeptide comprises the following amino acid sequence: (R¹)ₓ-Ser-Ala-Leu-Leu-Arg-Ser-Ile-Pro-Ala-(R²)_{y} (SEQ ID NO:20) and conservatively modified variations thereof. In this designation, R¹ denotes the orientation of the amino terminal (NH₂ or N-terminal) end and R² represents the orientation of the carboxyl terminal (COOH or C-terminal) end.

In the above formula, R¹ is an amino acid sequence comprising from 1 to about 40 amino acids, wherein each amino acid is independently selected from the group consisting of naturally occurring amino acids and amino acid analogs. The term “independently selected” is used herein to indicate that the amino acids making up the amino acid sequence R¹ may be identical or different (e.g., all of the amino acids in the amino acid sequence may be threonine, etc.). Moreover, as previously explained, the amino acids making up the amino acid sequence R¹ may be either naturally occurring amino acids, or known analogues of natural amino acids that functions in a manner similar to the naturally occurring amino acids (i.e., amino acid mimetics and analogs). Suitable amino acids that can be used to form the amino acid sequence R¹ include, but are not limited to, those listed in Table I, infra. The indexes “x” and “y” are independently selected and can be equal to one or zero.

As with R¹, R², in the above formula, is an amino acid sequence comprising from 1 to about 40 amino acids, wherein each amino acid is independently selected from the group consisting of naturally occurring amino acids and amino acid analogs. Moreover, as with R¹, the amino acids making up the amino acid sequence R² may be identical or different, and may be either naturally occurring amino acids, or known analogues of natural amino acids that functions in a manner similar to the naturally occurring amino acids (i.e., amino acid mimetics and analogs). Suitable amino acids that can be used to form R² include, but are not limited to, those listed in Table I, infra.

As used herein, “SAL” or “SAL peptide” refers to the formula above where x and y both equal 0. “SAL related peptide” refers to any of the other variants of SAL which are described the formula.

R¹ and R² are independently selected. If R¹R² are the same, they are identical in terms of both chain length and amino acid composition. Additional amino acids can be added to both the N-terminus and the C-terminus of the active peptide without loss of biological activity.

In another aspect, the present invention provides pharmaceutical compositions comprising one of the previously described SAL and SAL-related polypeptides and an appropriate combination therapeutic in a pharmaceutically acceptable diluent, carrier or excipient, in an amount sufficient to reduce symptoms of Alzheimer's disease, MS, or psychosis, including schizophrenia. In one embodiment, the SAL or SAL related peptide has an amino acid sequence selected from the group consisting of SEQ ID NOs:1 and 3-8, and conservatively modified variations thereof.

Known cell based assays can be used to assess the activity of a particular ADNF peptide. One method to determine biological activity of a NAP-like or SAL-like peptide mimetic is to assay their ability to protect neuronal cells from death. One such assay is performed using dissociated cerebral cortical cultures prepared as described (Brenneman & Gozes, J. Clin. Invest. 97:2299-2307 (1996)). The test paradigm consists of the addition of a test peptide to cultures that are co-treated with tetrodotoxin (TTX). TTX produces an apoptotic death in these cultures and, thus, is used as a model substance to demonstrate efficacy against this “programmed cell death” and all other means that produce this type of death mechanism. The duration of the test period is 5 days, and neurons are counted and identified by characteristic morphology and by confirmation with an immunocytochemical marker for neurons: e.g., neuron specific enolase.

The effect of an ADNF peptide on microtubules structure can be determined, using cell based assays or in vitro assays. Confocal microscopy can be used to assess microtubule structure in cells in the presence and absence of a tested ADNF peptide. Assays are disclosed e.g., in PCT/IL04/000232, filed Mar. 11, 2004, which is herein incorporated by reference for all purposes.

### III. Design and Synthesis of ADNF Polypeptides

Polypeptides and peptides comprising the core NAPVSIPQ (SEQ ID NO:2) or SALLRSIPA (SEQ ID NO:1) active site can be easily made, e.g., by systematically adding one amino acid at a time and screening the resulting peptide for biological activity, as described herein. In addition, the contributions made by the side chains of various amino acid residues in such peptides can be probed via a systematic scan with a specified amino acid, e.g., Ala.

One of skill will recognize many ways of generating alterations in a given nucleic acid sequence. Such well-known methods include site-directed mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques (see Giliman & Smith, Gene 8:81-97 (1979); Roberts et al., Nature 328:731-734 (1987)).

Most commonly, polypeptide sequences are altered by changing the corresponding nucleic acid sequence and expressing the polypeptide. However, polypeptide sequences are also optionally generated synthetically using commercially available peptide synthesizers to produce any desired polypeptide (see Merrifield, Am. Chem. Soc. 85:2149-2154 (1963); Stewart & Young, Solid Phase Peptide Synthesis (2nd ed. 1984)).

One of skill can select a desired nucleic acid or polypeptide of the invention based upon the sequences provided and upon knowledge in the art regarding proteins generally. Knowledge regarding the nature of proteins and nucleic acids allows one of skill to select appropriate sequences with activity similar or equivalent to the nucleic acids and polypeptides disclosed herein. The definitions section, supra, describes exemplar conservative amino acid substitutions.

Modifications to the NAP and ADNF polypeptides are evaluated by routine screening techniques in suitable assays for the desired characteristic. For instance, changes in the immunological character of a polypeptide can be detected by an appropriate immunological assay. Modifications of other properties such as nucleic acid hybridization to a target nucleic acid, redox or thermal stability of a protein, hydrophobicity, susceptibility to proteolysis, or the tendency to aggregate are all assayed according to standard techniques.

More particularly, it will be readily apparent to those of ordinary skill in the art that the small peptides of the present invention can readily be screened for ability to reduce symptoms of Alzheimer's disease or other tauopathy like progressive supranuclear palsy, MS, or psychosis, including schizophrenia by employing suitable cell-based assays and animal models known to those skilled in the art. Among the animal models employed to evaluate the cognition enhancing effects of drugs, the elevated plus-maze is probably the most popular (see Rodgers and Dalvi, Neurosci. Biobehay. Rev. 72:253-258 (1997) and Gozes et al., J Pharmacol Exp Ther.293(3):1091-1098 (2000)). Those in the art are aware that any of these standard behavioral models may be used to test NAP or ADNF polypeptides to identify or confirm reduction of symptoms of Alzheimer's disease or other tauopathy like progressive supranuclear palsy, MS, or psychosis, including schizophrenia.

Using these assays and models, one of ordinary skill in the art can readily prepare a large number of NAP and ADNF polypeptides in accordance with the teachings of the present invention and, in turn, screen them using the foregoing animal models to find ADNF polypeptides, in addition to those set forth herein, which possess the desired activity. For instance, using the NAP peptide (i.e., Asn-Ala-Pro-Val-Ser-Ile-Pro-Gln (SEQ ID NO:2)) or SAL peptide Ser-Ala-Leu-Leu-Arg-Ser-Ile-Pro-Ala (SEQ ID NO:1) as a starting point, one can systematically add, for example, Gly-, Gly-Gly-, Leu-Gly-Gly- to the N-terminus of the peptide and, in turn, screen each of these NAP or ADNF polypeptides in the foregoing assay to determine whether they possess biological activity. In doing so, it will be found that additional amino acids can be added to both the N-terminus and the C-terminus of the active site, i.e., Asn-Ala-Pro-Val-Ser-Ile-Pro-Gln (SEQ ID NO:2) or Ser-Ala-Leu-Leu-Arg-Ser-Ile-Pro-Ala (SEQ ID NO:1), without loss of biological activity.

The peptides of the invention may be prepared via a wide variety of well-known techniques. Peptides of relatively short size are typically synthesized on a solid support or in solution in accordance with conventional techniques (see, e.g., Merrifield, Am. Chem. Soc. 85:2149-2154 (1963)). Various automatic synthesizers and sequencers are commercially available and can be used in accordance with known protocols (see, e.g., Stewart & Young, Solid Phase Peptide Synthesis (2nd ed. 1984)). Solid phase synthesis in which the C-terminal amino acid of the sequence is attached to an insoluble support followed by sequential addition of the remaining amino acids in the sequence is the preferred method for the chemical synthesis of the peptides of this invention. Techniques for solid phase synthesis are described by Barany & Merrifield, Solid-Phase Peptide Synthesis; pp. 3-284 in The Peptides: Analysis, Synthesis, Biology. Vol. 2: Special Methods in Peptide Synthesis, Part A.; Merrifield et al., 1963; Stewart et al., 1984). NAP and related peptides are synthesized using standard Fmoc protocols (Wellings & Atherton, Methods Enzymol. 289:44-67 (1997)).

In addition to the foregoing techniques, the peptides for use in the invention may be prepared by recombinant DNA methodology. Generally, this involves creating a nucleic acid sequence that encodes the protein, placing the nucleic acid in an expression cassette under the control of a particular promoter, and expressing the protein in a host cell. Recombinantly engineered cells known to those of skill in the art include, but are not limited to, bacteria, yeast, plant, filamentous fungi, insect (especially employing baculoviral vectors) and mammalian cells.

The recombinant nucleic acids are operably linked to appropriate control sequences for expression in the selected host. For E. coli, example control sequences include the T7, trp, or lambda promoters, a ribosome binding site and, preferably, a transcription termination signal. For eukaryotic cells, the control sequences typically include a promoter and, preferably, an enhancer derived from immunoglobulin genes, SV40, cytomegalovirus, etc., and a polyadenylation sequence, and may include splice donor and acceptor sequences.

The plasmids of the invention can be transferred into the chosen host cell by well-known methods. Such methods include, for example, the calcium chloride transformation method for E. coli and the calcium phosphate treatment or electroporation methods for mammalian cells. Cells transformed by the plasmids can be selected by resistance to antibiotics conferred by genes contained on the plasmids, such as the amp, gpt, neo, and hyg genes.

Once expressed, the recombinant peptides can be purified according to standard procedures of the art, including ammonium sulfate precipitation, affinity columns, column chromatography, gel electrophoresis and the like (see, e.g., Scopes, Polypeptide Purification (1982); Deutscher, Methods in Enzymology Vol. 182: Guide to Polypeptide Purification (1990)). Once purified, partially or to homogeneity as desired, the NAP and ADNF polypeptides may then be used, e.g., to prevent neuronal cell death or as immunogens for antibody production. Optional additional steps include isolating the expressed protein to a higher degree, and, if required, cleaving or otherwise modifying the peptide, including optionally renaturing the protein.

After chemical synthesis, biological expression or purification, the peptide(s) may possess a conformation substantially different than the native conformations of the constituent peptides. In this case, it is helpful to denature and reduce the peptide and then to cause the peptide to re-fold into the preferred conformation. Methods of reducing and denaturing peptides and inducing re-folding are well known to those of skill in the art (see Debinski et al., J. Biol. Chem. 268:14065-14070 (1993); Kreitman & Pastan, Bioconjug. Chem. 4:581-585 (1993); and Buchner et al., Anal. Biochem. 205:263-270 (1992)). Debinski et al., for example, describe the denaturation and reduction of inclusion body peptides in guanidine-DTE. The peptide is then refolded in a redox buffer containing oxidized glutathione and L-arginine.

One of skill will recognize that modifications can be made to the peptides without diminishing their biological activity. Some modifications may be made to facilitate the cloning, expression, or incorporation of the targeting molecule into a fusion peptide. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

The ADNF peptides of the invention include ADNF peptides with non-natural amino acids, such as D amino acids. For the active core site sequences of ADNF I and ADNF II., one or more amino acids can be D-amino acids. In one embodiment, all the amino acids if SEQ ID NO:1, the ADNF I active core sequence, are D-amino acids. In another embodiment, all the amino acids if SEQ ID NO:2, the ADNF III active core sequence, are D-amino acids.

ADNF peptides also retain activity when conjugated to a lipophilic molecule, and in some cases have improved activities. Therefore, the invention also includes ADNF I and III peptides with lipophilic extensions.

ADNF peptides retain activity when conjugated to an ion chelator. Therefore, the invention also includes ADNF I and III peptides conjugated to an iron chelator. ADNF III peptides show enhanced antioxidant and anti-neurodegenerative activity when conjugated to ion chelators. See, e.g., Blat et al. J. Med. Chem. Dec. 14, 2007, e-publication; which is herein incorporated by reference for all purposes.

Another modification of ADNF peptides is conjugation to a femtomolar-acting humanin derivative named colivelin. Therefore, the invention also includes ADNF I and III peptides conjugated to colivin. ADNF III peptides show neuroprotective activity when conjugated to colivin. See, e.g., Chiba et al., J. Neurosci. 25:10252-10261 (2005); Yamada et al., Neuropsychopharmacology advance online publication, 10 Oct. 2007; doi:10.1038/sj.npp.1301591; and Arakawa et al., J. Peptide Sci. 2007 Nov. 12 (Epub ahead of print); each of which is herein incorporated by reference for all purposes.

### IV. ADNF Combination Treatment of Neurodegeneration

The present invention provides methods of treating neurodegeneration such as caused by dementia related to Alzheimer's disease, using a combination of an ADNF III polypeptide and a therapeutic used to treat the condition. The combination of the ADNF III polypeptide and the neurodegeneration therapeutic results in a treatment outcome that is improved as compared to treatment using only the ADNF peptide or only the neurodegeneration therapeutic.

Those of skill are aware of model systems that can be used for preliminary tests of the effectiveness of ADNF combination therapy against neurodegeneration, e.g., Alzheimer's disease and symptoms of the disease. One such model is the PDAPP mouse (platelet-derived growth factor promoter expressing amyloid precursor protein). The PDAPP mouse over expresses a human amyloid precursor protein with a substitution of phenylalanine for valine at amino acid 717. The PDAPP mouse progressively develops many of the neuropathological hallmarks of Alzheimer's disease in an age- and brain-region-dependent manner. See, e.g., Games et al., Nature, 373:523-527 (1995) and Johnson-Woods et al., Proc. Natl. Acad. Sci. USA, 94:1550-1555 (1997). The PDAPP mouse model has been used to test the effectiveness of Alzheimer's disease therapeutics, e.g., anti-amyloid vaccines. See, e.g., Schenk et al., Nature, 400:173-177 (1999). Other Alzheimer's models are described in, e.g., Bassan et al., 1999; Gozes et al., 2000; and Matsuoka et al., J Mol Neurosci. 2007; 31(2):165-70; each of which is herein incorporated by reference for all purposes. Alzheimer's disease is the most prevalent tauopathy. Other less abundant tauopathies can be also treated with an ADNF combination including models of frontotemporal dementia. See, e.g. Shiryaev et al., Neurobiol Dis. 34(2):381-388 (2009) and Ramsden et al., J Neurosci 25 (46): 10637-19647 (2005).

Those of skill are also aware of clinical tests that can be used to test human patients for reduction of neurodegeneration symptoms on receiving ADNF combination therapy, for example, ADAS-cog, Rosen et al., Am. J. Psychiat. 141:1356-1364 (1985); a neurophsychological test battery (NTB), Harrison et al., Arch. Neurol. 64:1323-1329 (2007); and an automated neuropsychological test battery (CANTAB), Egerhazi et al., Prog. Neuro-Psych & Biol. Psychia. 31:746-751 (2007). In some embodiments, improvement due to administration of ADNF combination therapy can be established when there is a statistically significant improvement in an individual patient compared to baseline or in a treated group versus a placebo group.

For ADNF combination therapy of neurodegeneration, the ADNF peptide can be a polypeptide comprising an ADNF III core active site peptide, also known as NAP. Examples of such peptides include a full-length ADNF III protein, e.g., a full-length human ADNF III protein; and SEQ ID NO:9-13. The polypeptide comprising an ADNF III core active site can include D-amino acid residues and in one embodiment, all of the ADNF III core active amino acid residues are D-amino acids. In some embodiments, the D-amino acid residues are found in the ADNF III core active site sequence. In another preferred embodiment, the ADNF peptide is the ADNF III core active site peptide, e.g., SEQ ID NO:2. The ADNF III core active site peptide can include one or more D-amino acid residues. In a further preferred embodiment, the ADNF III core active site peptide consists of all D-amino acid residues, i.e., SEQ ID NO:2 is all D-amino acids.

For ADNF combination therapy of neurodegeneration, the ADNF peptide can be a polypeptide comprising an ADNF I core active site peptide, also known as SAL. Examples of such peptides include a full-length ADNF I protein, e.g., a full-length human ADNF I protein; and SEQ ID NO:3-8. The polypeptide comprising an ADNF I core active site can include D-amino acid residues. In some embodiments, the D-amino acid residues are found in the ADNF I core active site sequence and in one embodiment, all of the ADNF I core active amino acid residues are D-amino acids. In another preferred embodiment, the ADNF peptide is the ADNF I core active site peptide, e.g., SEQ ID NO:1. The ADNF I core active site peptide can include one or more D-amino acid residues. In a further preferred embodiment, the ADNF I core active site peptide consists of all D-amino acid residues, i.e., SEQ ID NO:1 is all D-amino acids.

For ADNF combination therapy of neurodegeneration caused by, e.g., Alzheimer's disease, the ADNF peptide can be a mixture of polypeptide comprising an ADNF I core active site peptide, also known as SAL and a polypeptide comprising an ADNF III core active site peptide, also known as NAP. Examples of such peptides include a full-length ADNF I protein, e.g., a full-length human ADNF I protein; and SEQ ID NOs:3-8 and a full-length human ADNF III protein; and SEQ ID NOs:9-13: The polypeptide comprising an ADNF I core active site or the ADNF III core active site can include D-amino acid residues. In some embodiments, the D-amino acid residues are found in the ADNF I core active site sequence or the ADNF III core active site and in one embodiment, all of the ADNF I core active amino acid residues are D-amino acids or all of the ADNF III core active site amino acid residue are D-amino acids. The polypeptide comprising an ADNF I core active site and the ADNF III core active site can include D-amino acid residues. In some embodiments, the D-amino acid residues are found in the ADNF I core active site sequence and the ADNF III core active site and in one embodiment, all of the ADNF I core active amino acid residues are D-amino acids and all of the ADNF III core active site amino acid residue are D-amino acids. In another preferred embodiment, the ADNF I peptide in the ADNF mixture is the ADNF I core active site peptide, e.g., SEQ ID NO:1. The ADNF I core active site peptide can include one or more D-amino acid residues. In a further preferred embodiment, the ADNF I core active site peptide consists of all D-amino acid residues, i.e., SEQ ID NO:1 is all D-amino acids. In another preferred embodiment, the ADNF III peptide in the ADNF mixture is the ADNF III core active site peptide, e.g., SEQ ID NO:2. The ADNF III core active site peptide can include one or more D-amino acid residues. In a further preferred embodiment, the ADNF III core active site peptide consists of all D-amino acid residues, i.e., SEQ ID NO:2 is all D-amino acids.

The neudegeneration therapeutic is preferably selected from one of the following classes of compounds: an acetylcholinesterase inhibitor, an N-methyl D-aspartate (NMDA) receptor antagonist, an amyloid vaccine, an amyloid-reducing agent, an ion chelating agent, a neuroprotective agent, an anti-inflammatory agent or antioxidant, an inhibitors of tau phosphorylation, a muscarinic agonist, a nicotinic interacting compound, or a neurotransmitter modulator. Combinations of agents from one or more classes can be used or combination of agents from a single class can also be used.

For ADNF combination therapy of neurodegeneration such as Alzheimer's disease, an acetylcholinesterase inhibitor is selected from huperzine, metrifonate, physostigmine, neostigmine, pyridostigmine, ambenonium, demarcarium, rivastigmine, galantamine, donepezil, Tacrine, Edrophonium, and phenothiazine.

For ADNF combination therapy of neurodegeneration such as Alzheimer's disease, an exemplary NMDA receptor antagonist is memantine. Other NMDA antagonists can be used including uncompetitive channel blockers, such as Amantadine, Dextromethorphan, Dextrorphan, Ibogaine, Ketamine, Nitrous oxide, Phencyclidine, Riluzole, Tiletamine, and Ethanol in high doses. Useful noncompetitive antagonists include Dizocilpine, Aptiganel, and Memantine (Axura®, Akatinol®, Namenda®, Ebixa®, 1-amino-3,5-dimethylada-mantane), and Remacimide.

For ADNF combination therapy of neurodegeneratio such as Alzheimer's disease, an exemplary amyloid vaccine is directed against aggregated amyloid plaques, fragments of amyloid peptides, conformational epitopes of amyloid proteins, or memapsin 2. Vaccines can be administered using either active immunization or passive immunization by infusion of antibodies raised against these epitopes/antigens.

For ADNF combination therapy of neurodegeneration such as Alzheimer's disease, an amyloid-reducing agent is selected from an amyloid aggregate breaking agent, an inhibitor of aggregate formation, a BAC1 inhibitor, a gamma secretase inhibitor, or an ADAM 10 activator. Furthermore, therapeutic agents known to inhibit tau aggregation such as methylene blue (Rember), phenylthiazolyl-hydrazide (PTH), and aminothienopyridazines (ATPZs) are also useful for the ADNF combination use of this invention to treat or prevent neurodegeneration caused by a tauopathy-related dementia.

For ADNF combination therapy of neurodegeneration such as Alzheimer's disease, exemplary ion chelating agents are ethylenediamine, ethylenediaminetetraacetic acid (EDTA), heme, Dimercaprol (2,3-dimercapto-1-propanol), Porphine, Desfuroxamine Mesylate: used for iron toxicity, DMSA: an analogue of Dimercaprol, D-penicillamine: an oral chelating agent used for lead, arsenic, or mercury poisoning, and Calcium Disodium Versante (CaNa₂-EDTA). In some embodiments, chelating agents are conjugated to an ADNF peptide. See, e.g., Blat et al. J. Med. Chem. Dec. 14, 2007, e-publication; which is herein incorporated by reference for all purposes.

For ADNF combination therapy of neurodegeneration such as Alzheimer's disease, a neuroprotective agent is selected from cerebrolysine, nerve growth factor, cell-based therapy, and selenite.

For ADNF combination therapy of neurodegeneration such as Alzheimer's disease, an anti-inflammatory agent or antioxidant. An antioxidant is selected from vitamin E and Gingko biloba. An anti-inflammatory agent is selected from ibuprofen, flubiprofen, indomethacin, ketoprofen, diclofenac, meloxicam, R-flubiprofen, naproxen, rofecoxib, valdecoxib, zomepirac, etodolac, etoricoxib, parecoxib, celecoxib, sulindac, mesalazine, sulfasalazine, and ethenzamide.

For ADNF combination therapy of neurodegeneration such as Alzheimer's disease, an inhibitor of tau phosphorylation is a GSK inhibitor selected from 4-Benzyl-2-methyl-1,2,4-thiadiazolidine-3,5-dione (TDZD-8), AR— A014418, 4-Acylamino-6-arylfuro&lsqb; 2,3-d&rsqb; pyrimidines, lithium, SB-415286, P24, CT98014, and CHIR98023.

For ADNF combination therapy of neurodegeneration such as Alzheimer's disease, a muscarinic agonist is, e.g., Cevimeline (AF102B) (Evoxac™) and AF267B, which is disclosed at Neuron. March 2; 49(5):671-82 (2006).

For ADNF combination therapy of neurodegeneration such as Alzheimer's disease, a nicotinic interacting compound is selected from N-[(7S,8S)-7-(pyridin-3-ylmethyl)-1-azabicyclo[2.2.2]octan-8-yl]-1-benzofuran-2-carboxamide, ABT 107, 3-[(3E)-3-[(2,4-dimethoxyphenyemethylidene]-5,6-dihydro-4H-pyridin-2-yl]pyridine dihydrochloride, EVP6124, and MEM 3454/R1589. Dimebolin hydrochloride (brand name dimebon), a neuroprotective agent, may also be used in ADNF combination theurapy. See, e.g., WO2008/051599.

In a preferred embodiment, combination therapy of neurodegeneration such as Alzheimer's disease is performed using a combination of a polypeptide comprising an ADNF III active core site and an acetylcholinesterase inhibitor selected from huperzine, metrifonate, physostigmine, neostigmine, pyridostigmine, ambenonium, demarcarium, rivastigmine, galantamine, donepezil, Tacrine, Edrophonium, and phenothiazine. In a further preferred embodiment, the ADNF III active core site peptide consists of all D-amino acids.

In a preferred embodiment, combination therapy of neurodegeneration such as Alzheimer's disease is performed using a combination of a polypeptide comprising an ADNF I active core site and an acetylcholinesterase inhibitor selected from huperzine, metrifonate, physostigmine, neostigmine, pyridostigmine, ambenonium, demarcarium, rivastigmine, galantamine, donepezil, Tacrine, Edrophonium, and phenothiazine. In a further preferred embodiment, the ADNF I active core site peptide consists of all D-amino acids.

In some embodiments, combination therapy of neurodegeneration such as Alzheimer's disease is performed sequentially. For example, the ADNF polypeptide can be administered a certain time before or after the neurodegeneration therapeutic. Alternatively, the ADNF polypeptide can be administered upon the detection of a therapeutic effect of the neurodegeneration therapeutic on the patient. The neurodegeneration therapeutic can also be administered upon the detection of a therapeutic effect of the ADNF polypeptide. In some embodiments, the ADNF polypeptide is administered at the same time as the neurodegeneration therapeutic. In some embodiments, combination therapy is performed such that administration of the ADNF polypeptide follows a first regime, while administration of the neurodegeneration therapeutic follows a second regime.

### V. ADNF Combination Treatment of Multiple Sclerosis

The present invention provides methods of treating multiple sclerosis (MS) using a combination of an ADNF III polypeptide and a therapeutic used to treat MS. The combination of the ADNF III polypeptide and the MS therapeutic results in a treatment outcome that is improved as compared to treatment using only the ADNF peptide or only the MS therapeutic.

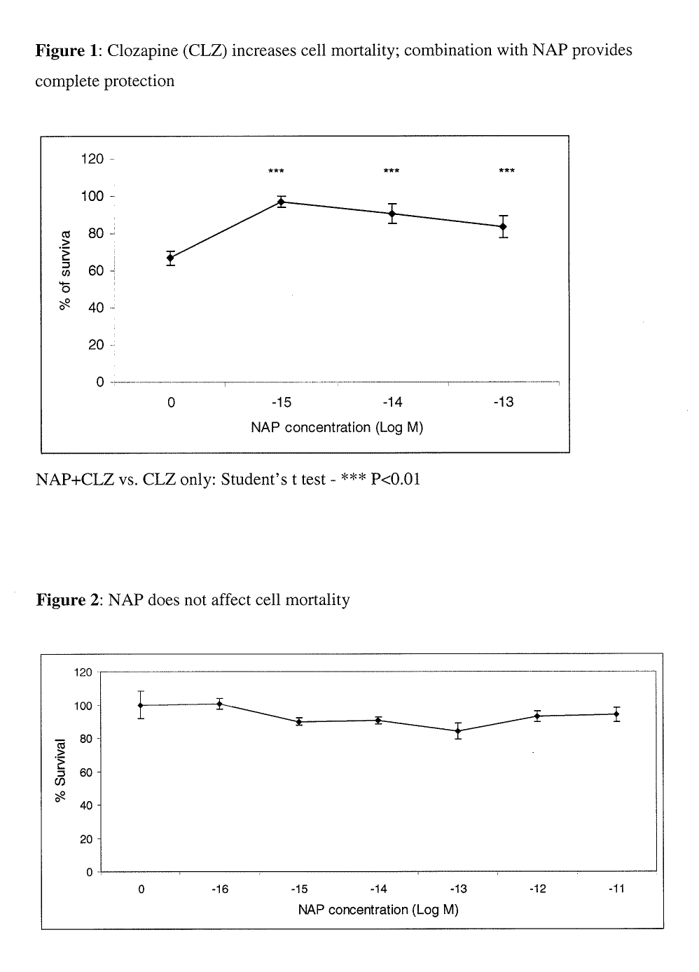

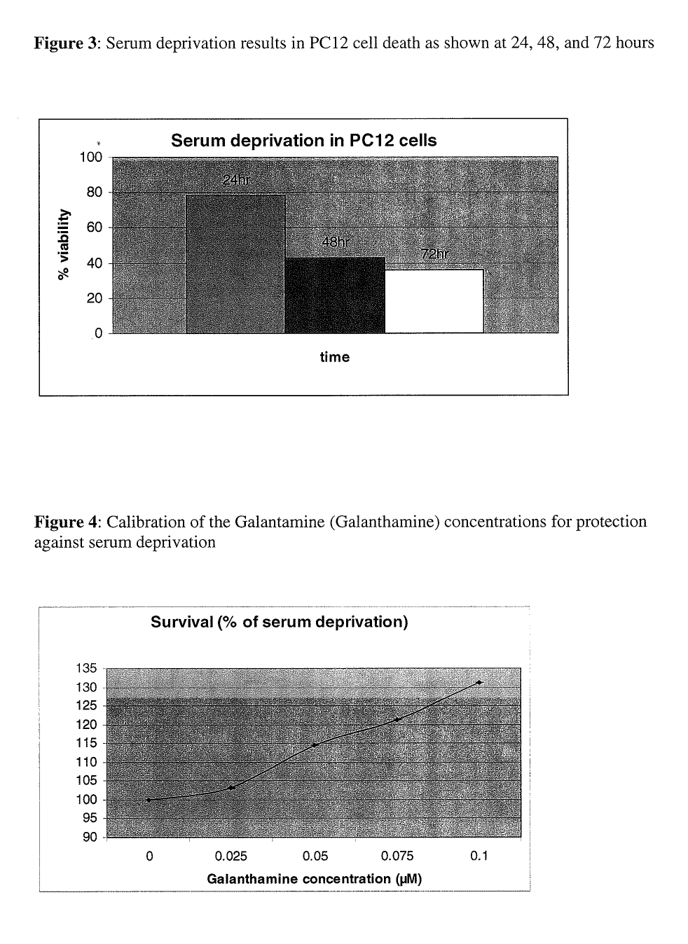

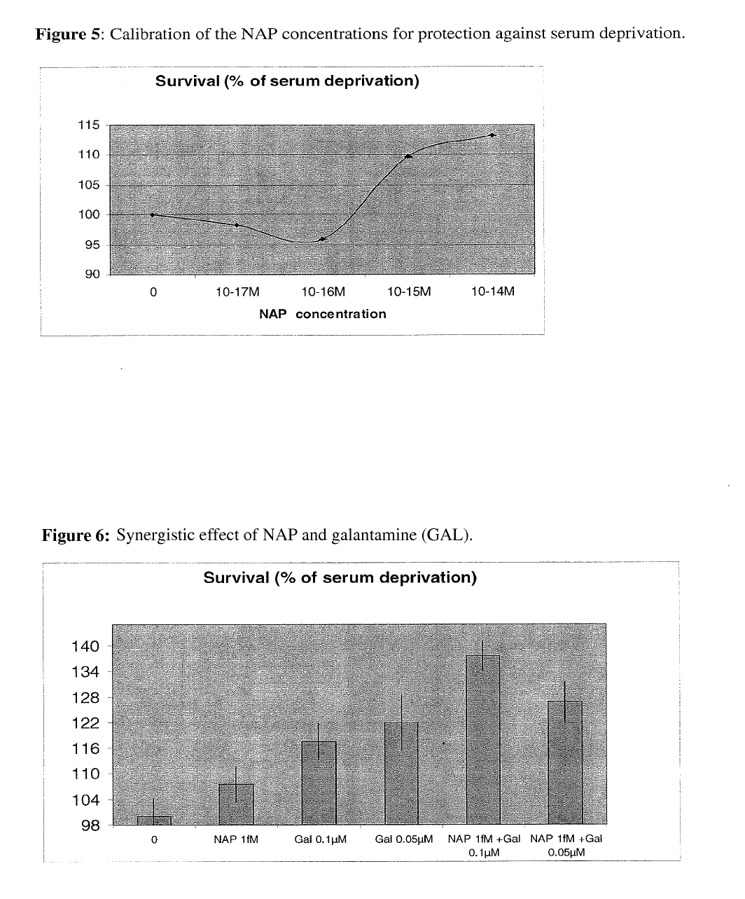

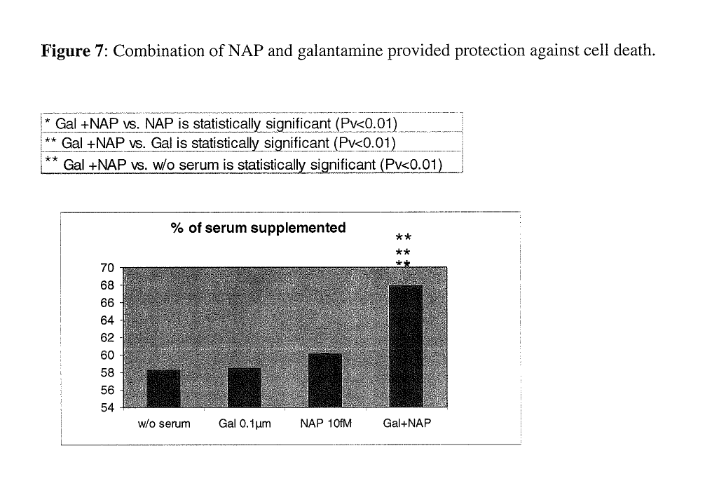

Those of skill are aware of model systems that can be used for preliminary tests of the effectiveness of ADNF combination therapy against MS and symptoms of the disease. One such model is the myelin-oligodedrocyte glycoprotein (MOG)-induced chronic experimental autoimmune encephalomyelitis (EAE) mouse model. To induce MS-like symptoms of axonal damage and demyelination, EAE was induced by immunization of the animal with a peptide encompassing amino acids 35-55 of rat MOG. A previous study by Offen et al. investigated the possible role of axonal susceptibility and resistance to reactive oxygen species (ROS) in the pathogenesis of EAE. See, e.g., Offen et al. J Mol Neurosci. 15(3):167-76 (2000). Offen et al. demonstrated that clinical manifestation of MS were apparent in the MOG-induced chronic EAE model. Clinical manifestations included lose of tail tonicity, partial hind-limb paralysis, and complete hind-limb paralysis. See, e.g., Offen et al., FIG. 1, page 170. Offen et al. also demonstrated that after immunization with MOG, the immune response to the protein included T cell proliferation. See, e.g., Offen et al., FIG. 3, page 172.

Those of skill are also aware of clinical tests that can be used to test human patients for reduction of MS symptoms on receiving ADNF combination therapy. Effective treatment of multiple sclerosis may be examined in several different ways. Satisfying any of the following criteria evidences effective treatment. Three main criteria are used: EDSS (extended disability status scale), appearance of exacerbations or MRI (magnetic resonance imaging).

The EDSS is a means to grade clinical impairment due to MS. See, e.g., Kurtzke, Neurology 33:1444 (1983). Eight functional systems are evaluated for the type and severity of neurologic impairment. Briefly, prior to treatment, patients are evaluated for impairment in the following systems: pyramidal, cerebella, brainstem, sensory, bowel and bladder, visual, cerebral, and other. Follow-ups are conducted at defined intervals. The scale ranges from 0 (normal) to 10 (death due to MS). In some embodiments, a decrease of at least one full step represents an effective treatment in the context of the present invention. See, e.g., Kurtzke, Ann. Neurol. 36:573-79 (1994).

Exacerbations are defined as the appearance of a new symptom that is attributable to MS and accompanied by an appropriate new neurologic abnormality (IFNB MS Study Group, supra). In addition, the exacerbation must last at least 24 hours and be preceded by stability or improvement for at least 30 days. Briefly, patients are given a standard neurological examination by clinicians. Exacerbations are either mild, moderate, or severe according to changes in a Neurological Rating Scale. See, e.g., Sipe et al., Neurology 34:1368 (1984). An annual exacerbation rate and proportion of exacerbation-free patients are determined. In some embodiments, therapy is effective if there is a statistically significant difference in the rate or proportion of exacerbation-free patients between the treated group and the placebo group (or for a single subject, after treatment with an ADNF III polypeptide compared to before the subject was treated) for either of these measurements. In addition, time to first exacerbation and exacerbation duration and severity may also be measured. In some embodiments, a measure of effectiveness using an ADNF III polypeptide in this regard is a statistically significant difference in the time to first exacerbation or duration and severity in the treated group compared to a control group.

MRI can be used to measure active lesions using gadolinium-DTPA-enhanced imaging or the location and extent of lesions using T2-weighted techniques. See, e.g., McDonald et al. Ann. Neurol. 36:14, (1994). Briefly, baseline MRIs are obtained. The same imaging plane and patient position are used for each subsequent study. Positioning and imaging sequences are chosen to maximize lesion detection and facilitate lesion tracing. The same positioning and imaging sequences are used on subsequent studies. The presence, location, and extent of MS lesions are determined by radiologists. Areas of lesions are outlined and summed slice by slice for total lesion area. At least three aspects can be examined: evidence of new lesions, rate of appearance of active lesions, percentage change in lesion area. See, e.g., Paty et al., Neurology 43:665, (1993). In some embodiments, improvement due to administration of ADNF combination therapy can be established when there is a statistically significant improvement in an individual patient compared to baseline or in a treated group versus a placebo group.

Efficacy of the peptide analogue in the context of prevention is judged based on the following criteria: frequency of myelin basic protein (MBP)-reactive T-cells determined by limiting dilution, proliferation response of MBP-reactive T-cell lines and clones, and cytokine profiles of T-cell lines and clones to MBP established from patients. Effective doses can decrease the frequency of reactive cells, reduce proliferation of MBP-reactive T-cells, and/or reduce levels of TNF and IFN-α. Quintana et al., Ann NY Acad. Sci. 1070:500-506 (2006) reported that the ADNF III core peptide, also known as NAP (SEQ ID NO:2), downregulates the key inflammatory cytokines TNF-α, interleukin-16 (IL-16), and IL-12 in macrophages. Braitch et al., Neuroimmunomodulation 17(2):120-125 (2009) discussed the immunomodulatory effect of the ADNF III peptide, also known as NAP (SEQ ID NO:2). The effect on T-cell activation was conducted by measuring the surface expression of CD69 and CD154 and its effect on the expression of the pro-inflammatory cytokine, interferon-gamma (IFN-γ). Treatment with the ADNF III peptide decreased the expression of CD69, CD154 and IFN-γ in peripheral blood mononuclear cells (PBMCs, i.e., T cells, B cells, monocytes, and natural killer cells). The ADNF III peptide suppressed the anti-CD3-/anti-CD28-stimulated proliferation of PBMCs. Clinical measurements include the relapse rate in one and two year intervals, and a change in EDSS, including time to progression from baseline of 1.0 unit on the EDSS which persists for six months. On a Kaplan-Meier curve, a delay in sustained progression of disability shows efficacy. Other criteria include a change in area and volume of T2 images on MRI, and the number and volume of lesions determined by gadolinium enhanced images.

For ADNF combination therapy of MS, the ADNF peptide can be a polypeptide comprising an ADNF III core active site peptide, also known as NAP. Examples of such peptides include a full-length ADNF III protein, e.g., a full-length human ADNF III protein; and SEQ ID NO:9-13. The polypeptide comprising an ADNF III core active site can include D-amino acid residues and in one embodiment, all of the ADNF III core active amino acid residues are D-amino acids. In some embodiments, the D-amino acid residues are found in the ADNF III core active site sequence. In another preferred embodiment, the ADNF peptide is the ADNF III core active site peptide, e.g., SEQ ID NO:2. The ADNF III core active site peptide can include one or more D-amino acid residues. In a further preferred embodiment, the ADNF III core active site peptide consists of all D-amino acid residues, i.e., SEQ ID NO:2 is all D-amino acids.

For ADNF combination therapy of MS, the ADNF peptide can be a polypeptide comprising an ADNF I core active site peptide, also known as SAL. Examples of such peptides include a full-length ADNF I protein, e.g., a full-length human ADNF I protein; and SEQ ID NO:3-8. The polypeptide comprising an ADNF I core active site can include D-amino acid residues. In some embodiments, the D-amino acid residues are found in the ADNF I core active site sequence and in one embodiment, all of the ADNF I core active amino acid residues are D-amino acids. In another preferred embodiment, the ADNF peptide is the ADNF I core active site peptide, e.g., SEQ ID NO:1. The ADNF I core active site peptide can include one or more D-amino acid residues. In a further preferred embodiment, the ADNF I core active site peptide consists of all D-amino acid residues, i.e., SEQ ID NO:1 is all D-amino acids.

For ADNF combination therapy of MS, the ADNF peptide can be a mixture of polypeptide comprising an ADNF I core active site peptide, also known as SAL and a polypeptide comprising an ADNF III core active site peptide, also known as NAP. Examples of such peptides include a full-length ADNF I protein, e.g., a full-length human ADNF I protein; and SEQ ID NOs:3-8 and a full-length human ADNF III protein; and SEQ ID NOs:9-13. The polypeptide comprising an ADNF I core active site or the ADNF III core active site can include D-amino acid residues. In some embodiments, the D-amino acid residues are found in the ADNF I core active site sequence or the ADNF III core active site and in one embodiment, all of the ADNF I core active amino acid residues are D-amino acids or all of the ADNF III core active site amino acid residue are D-amino acids. The polypeptide comprising an ADNF I core active site and the ADNF III core active site can include D-amino acid residues. In some embodiments, the D-amino acid residues are found in the ADNF I core active site sequence and the ADNF III core active site and in one embodiment, all of the ADNF I core active amino acid residues are D-amino acids and all of the ADNF III core active site amino acid residue are D-amino acids. In another preferred embodiment, the ADNF I peptide in the ADNF mixture is the ADNF I core active site peptide, e.g., SEQ ID NO:1. The ADNF I core active site peptide can include one or more D-amino acid residues. In a further preferred embodiment, the ADNF I core active site peptide consists of all D-amino acid residues, i.e., SEQ ID NO:1 is all D-amino acids. In another preferred embodiment, the ADNF III peptide in the ADNF mixture is the ADNF III core active site peptide, e.g., SEQ ID NO:2. The ADNF III core active site peptide can include one or more D-amino acid residues. In a further preferred embodiment, the ADNF III core active site peptide consists of all D-amino acid residues, i.e., SEQ ID NO:2 is all D-amino acids.

For ADNF combination therapy of MS, the MS therapeutic is preferably selected from the following: glatiramer acetate (GA), also known as copolymer-1, and an interferon beta protein.

GA has been shown to be effective in treating MS. Daily subcutaneous injections of glatiramer acetate (20 mg/injection) reduce relapse rates, progression of disability, appearance of new lesions by magnetic resonance imaging (MRI) and appearance of “black holes.” See, e.g., Johnson, et al., Neurol., 45:1268 (1989) and Filippi, et al., Neurol. 57:731-733 (2001).

In rodents, GA suppresses the encephalitogenic effects of auto reactive T-cells. Passive transfer of GA-reactive T-cells prevents the development of EAE induced in rats or mice by MBP, protolipid protein (PLP) or Myelin Oligodendrocyte Glycoprotein (MOG). In humans, daily injection of GA, resulted in the development of a T helper 2 (Th2)-type of protective response over time. These activated GA-reactive T-cells, when reaching the site of injury, secrete cytokines associated with both Th2 (IL-4) profiles and neurotrophic factors such as Brain Derived Neurotrophic Factor (BDNF, Ziemssen and Schrempf. Int Rev Neurobiol.; 79:537-70 (2007) Review) serves a dual role: first exerting bystander suppression anti-inflammatory activity and later a neuroprotective action on axons.

At least two forms of interferon beta are used to treat MS in humans, e.g., interferon beta-1a, AVONEX® or REBIF®; and interferon beta-1b, BETASERON®.

In another preferred embodiment, combination therapy of MS is performed using a combination of an ADNF III active core site and GA. In a further preferred embodiment, the ADNF III active core site peptide consists of all D-amino acids. In other embodiments, the ADNF III polypeptide comprising the active core site is used in combination with interferon β-1b (tradename Betaferon or Betaseron), an interferon known for its use in treating various forms of MS.

In a preferred embodiment, combination therapy of MS is performed using a combination of a polypeptide comprising an ADNF I active core site and GA. In a further preferred embodiment, the ADNF I active core site peptide consists of all D-amino acids. In other embodiments, the ADNF I polypeptide comprising the active core site is used in combination with interferon β-1b.

In some embodiments, combination therapy of MS is performed sequentially. For example, the ADNF polypeptide can be administered at a certain time before or after the MS therapeutic. Alternatively, the ADNF polypeptide can be administered upon the detection of a therapeutic effect of the MS therapeutic on the patient. The MS therapeutic can also be administered upon the detection of a therapeutic effect of the ADNF polypeptide. In some embodiments, the ADNF polypeptide is administered at the same time as the MS therapeutic. In some embodiments, combination therapy is performed such that administration of the ADNF polypeptide follows a first regime, while administration of the MS therapeutic follows a second regime.

### VI. ADNF Combination Treatment of Schizophrenia

The present invention provides methods of treating psychosis, in particular schizophrenia, using a combination of an ADNF III polypeptide and a therapeutic used to treat psychosis, particularly schizophrenia. The combination of the ADNF III polypeptide and the psychosis or schizophrenia therapeutic results in a treatment outcome that is improved as compared to treatment using only the ADNF peptide or only the psychosis or schizophrenia therapeutic.

Those of skill are aware of model systems that can be used for preliminary tests of the effectiveness of ADNF combination therapy against psychosis, particularly schizophrenia. One such model is the STOP protein-deficient mouse model of schizophrenia See, e.g., Andrieux et al., Genes & Develop., 16:2350-2364 (2002). STOP proteins are calmodulin-binding and calmodulin-regulated microtubule associated proteins (MAPS). Neurons express STOP proteins. Using antibodies directed against the STOP protein in cell based assays, inhibition of STOP protein activity suppressed microtubule cold stability. In cultured neuronal cells, STOP protein inhibition also inhibited neuronal differentiation. Andrieux et al. used gene targeting to knock out the STOP protein of mice. Microtubules derived from cells of the STOP protein-deficient mice had a dramatic loss of stability as compared to microtubules from unmodified control mice. The STOP protein-deficient mice had apparently normal brain histology, but had synaptic abnormalities. See, e.g., Andrieux et al., page 2351. Andrieux et al. also analyzed the behavior of the STOP protein deficient mice. The STOP protein-deficient mice exhibited atypical behavior, including phases of intense activity without apparent goal orientation, and random shifts between activities. The STOP protein-deficient mice exhibited crisis behavior, lasting over 20 minutes, with continuous burrowing or cage circling behavior. These behaviors were never observed in wild-type-control mice. See, Andrieux, et al., page 2355. Andrieux et al. also evaluated the STOP protein-deficient mice for ability to perform major classes of behavioral tasks. The STOP protein-deficient mice exhibited dramatic anxiety-like behavior, defects in short-term memory and learning, as well as severe social withdrawal. See, e.g., Andrieux et al., at page 2357. Finally, STOP protein-deficient female mice failed to nurture offspring without intervention, resulting in the death of their pups. See, e.g., Andrieux et al., at page 2358. Andrieux et al. recognized that the behavioral defects of the STOP protein-deficient mice were reminiscent of schizophrenia models and tested the effect of known schizophrenia treatments on that behavior. Andrieux et al. treated STOP protein-deficient female mice with neuroleptics over four months, including a pregnancy, delivery, and post-partum period. Neuroleptics are used to treat schizophrenia in patients, including human patients. The neuroleptic treatment resulted in survival of some of the pups, compared to no surviving pups from the untreated female STOP protein-deficient mice. See, e.g., Andrieux et al., page 2358.

Those of skill are also aware of clinical tests that can be used to test human patients for reduction of psychotic symptoms on receiving ADNF combination therapy and in particular for reduction of symptoms of schizophrenia. The ADNF combination that exhibits the anti-psychotic or anti-schizophrenic activity of interest or which provides either a subjective relief of a symptom(s) or an objectively identifiable improvement as noted by the clinician or other qualified observer.

For ADNF combination therapy of psychosis, particularly schizophrenia, the ADNF peptide can be a polypeptide comprising an ADNF III core active site peptide, also known as NAP. Examples of such peptides include a full-length ADNF III protein, e.g., a full-length human ADNF III protein; and SEQ ID NOs:9-13. The polypeptide comprising an ADNF III core active site can include D-amino acid residues and in one embodiment, all of the ADNF III core active amino acid residues are D-amino acids. In some embodiments, the D-amino acid residues are found in the ADNF III core active site sequence. In another preferred embodiment, the ADNF peptide is the ADNF III core active site peptide, e.g., SEQ ID NO:2. The ADNF III core active site peptide can include one or more D-amino acid residues. In a further preferred embodiment, the ADNF III core active site peptide consists of all D-amino acid residues, i.e., SEQ ID NO:2 is all D-amino acids.

For ADNF combination therapy of psychosis, particularly schizophrenia, the ADNF peptide can be a polypeptide comprising an ADNF I core active site peptide, also known as SAL. Examples of such peptides include a full-length ADNF I protein, e.g., a full-length human ADNF I protein; and SEQ ID NOs:3-8. The polypeptide comprising an ADNF I core active site can include D-amino acid residues. In some embodiments, the D-amino acid residues are found in the ADNF I core active site sequence and in one embodiment, all of the ADNF I core active amino acid residues are D-amino acids. In another preferred embodiment, the ADNF peptide is the ADNF I core active site peptide, e.g., SEQ ID NO:1. The ADNF I core active site peptide can include one or more D-amino acid residues. In a further preferred embodiment, the ADNF I core active site peptide consists of all D-amino acid residues, i.e., SEQ ID NO:1 is all D-amino acids.

For ADNF combination therapy of psychosis, particularly schizophrenia, the ADNF peptide can be a mixture of polypeptide comprising an ADNF I core active site peptide, also known as SAL and a polypeptide comprising an ADNF III core active site peptide, also known as NAP. Examples of such peptides include a full-length ADNF I protein, e.g., a full-length human ADNF I protein; and SEQ ID NOs:3-8; and a full-length human ADNF III protein; and SEQ ID NOs:9-13. The polypeptide comprising an ADNF I core active site or the ADNF III core active site can include D-amino acid residues. In some embodiments, the D-amino acid residues are found in the ADNF I core active site sequence or the ADNF III core active site and in one embodiment, all of the ADNF I core active amino acid residues are D-amino acids or all of the ADNF III core active site amino acid residue are D-amino acids. The polypeptide comprising an ADNF I core active site and the ADNF III core active site can include D-amino acid residues. In some embodiments, the D-amino acid residues are found in the ADNF I core active site sequence and the ADNF III core active site and in one embodiment, all of the ADNF I core active amino acid residues are D-amino acids and all of the ADNF III core active site amino acid residue are D-amino acids. In another preferred embodiment, the ADNF I peptide in the ADNF mixture is the ADNF I core active site peptide, e.g., SEQ ID NO:1. The ADNF I core active site peptide can include one or more D-amino acid residues. In a further preferred embodiment, the ADNF I core active site peptide consists of all D-amino acid residues, i.e., SEQ ID NO:1 is all D-amino acids. In another preferred embodiment, the ADNF III peptide in the ADNF mixture is the ADNF III core active site peptide, e.g., SEQ ID NO:2. The ADNF III core active site peptide can include one or more D-amino acid residues. In a further preferred embodiment, the ADNF III core active site peptide consists of all D-amino acid residues, i.e., SEQ ID NO:2 is all D-amino acids.

Conventional antipsychotics, also termed “typical antipsychotics” are effective in improving symptoms of schizophrenia and are characterized by their antagonist affinity for the D₂ dopamine receptor. This pharmacological effect results in acutely diminished activity of the brain's dopamine neurotransmitter systems. Conventional antipsychotics can be classified into high, medium and low potency based on their proportional affinity for the D₂ receptor. Conventional or “typical” antipsychotics include chlorpromazine, fluphenazine, haloperidol, loxapine, mesoridazine, molindone perphenazine, pimozide, thioridazine, thioxthixene, trifluoperidone (The Merck Manual of Diagnosis and Therapy, 17^{th} Edition, pp 1563-1573, 1999). Significant numbers of patients suffering from schizophrenia and other psychoses have proven resistant to treatment with conventional antipsychotics. Moreover, conventional antipsychotics produce movement related adverse effects related to disturbances in the nigrostriatal dopamine system. These extrapyramidal side effects (EPS) include Parkinsonism, akathisia, tardive dyskinesia and dystonia.

“Atypical” antipsychotics refer to antipsychotic drugs that produce antipsychotic effects with little or no EPS and include clozapine, risperidone, olanzapine, quetiapine, ziprasidone and aripiprazole. “Atypical” antipsychotics differ from conventional antipsychotics in their pharmacological profiles. While conventional antipsychotics are characterized principally by D₂ dopamine receptor blockade, atypical antipsychotics show antagonist effects on multiple receptors including the 5HT₂ₐ and 5HT_{2c} serotonin receptors and varying degrees of receptor affinities. Atypical antipsychotic drugs are commonly referred to as serotonin/dopamine antagonists, reflecting the influential hypothesis that greater affinity for the 5HT₂ receptor than for the D₂ receptor underlies “atypical” antipsychotic drug action or “second generation” antipsychotic drugs.

For ADNF combination therapy of psychosis, particularly schizophrenia, the psychosis or schizophrenia therapeutic is preferably selected from the following: Aripiprazole (ABILIFY®), Clozapine (CLOZARIL®), Ziprasidone (GEODON®), Resperidone (RISPERDAL®), Quetiapine (SEROQUEL®), Olanzapine (ZYPREXA®), asenapine, iloperidone, and bifeprunox.

In another preferred embodiment, combination therapy of psychosis, particularly schizophrenia, is performed using a combination of an ADNF III active core site and a psychosis or schizophrenia therapeutic selected from Aripiprazole, Clozapine, Ziprasidone, Resperidone, Quetiapine, and Olanzapine. In a further preferred embodiment, the ADNF III active core site peptide consists of all D-amino acids.

In a preferred embodiment, combination therapy of psychosis, particularly schizophrenia, is performed using a combination of a polypeptide comprising an ADNF I active core site and a psychosis or schizophrenia therapeutic selected from Aripiprazole, Clozapine, Ziprasidone, Resperidone, Quetiapine, and Olanzapine. In a further preferred embodiment, the ADNF I active core site peptide consists of all D-amino acids.

In some embodiments, combination therapy of psychosis, particularly schizophrenia, is performed sequentially. For example, the ADNF polypeptide can be administered a certain time before or after the psychosis or schizophrenia therapeutic. Alternatively, the ADNF polypeptide can be administered upon the detection of a certain therapeutic effect of the psychosis or schizophrenia therapeutic on the patient. The psychosis or schizophrenia therapeutic can also be administered upon the detection of a certain therapeutic effect of the ADNF polypeptide. In some embodiments, the ADNF polypeptide is administered at the same time as the psychosis or schizophrenia therapeutic. In some embodiments, combination therapy is performed such that administration of the ADNF polypeptide follows a first regime, while administration of the psychosis or schizophrenia therapeutic follows a second regime.

### VII. Pharmaceutical Administration

The pharmaceutical compositions of the present invention are suitable for use in a variety of drug delivery systems. Peptides that have the ability to cross the blood brain barrier can be administered, e.g., systemically, nasally, etc., using methods known to those of skill in the art. Larger peptides that do not have the ability to cross the blood brain barrier can be administered to the mammalian brain via intracerebroventricular (ICV) injection or via a cannula using techniques well known to those of skill in the art (see, e.g., Motta & Martini, Proc. Soc. Exp. Biol. Med. 168:62-64 (1981); Peterson et al., Biochem. Pharamacol. 31:2807-2810 (1982); Rzepczynski et al., Metab. Brain Dis. 3:211-216 (1988); Leibowitz et al., Brain Res. Bull. 21:905-912 (1988); Sramka et al., Stereotact. Funct. Neurosurg. 58:79-83 (1992); Peng et al., Brain Res. 632:57-67 (1993); Chem et al., Exp. Neurol. 125:72-81 (1994); Nikkhah et al., Neuroscience 63:57-72 (1994); Anderson et al., J. Comp. Neurol. 357:296-317 (1995); and Brecknell & Fawcett, Exp. Neurol. 138:338-344 (1996)).

Suitable formulations for use in the present invention are found in Remington's Pharmaceutical Sciences (17th ed. 1985)), which is incorporated herein by reference. In addition, for a brief review of methods for drug delivery, see Langer, Science 249:1527-1533 (1990), which is incorporated herein by reference. Suitable dose ranges are described in the examples provided herein, as well as in WO 9611948, herein incorporated by reference in its entirety.

As such, the present invention provides for therapeutic compositions or medicaments comprising one or more of the NAP or ADNF polypeptides described hereinabove in combination with a pharmaceutically acceptable excipient, wherein the amount of the NAP or ADNF polypeptide is sufficient to provide a therapeutic effect.

In a therapeutic application, the NAP and ADNF polypeptides of the present invention are embodied in pharmaceutical compositions intended for administration by any effective means, including parenteral, topical, oral, pulmonary (e.g., by inhalation) or local administration. Preferably, the pharmaceutical compositions are administered parenterally, e.g., intravenously, subcutaneously, intradermally, or intramuscularly, or intranasally.

Thus, the invention provides compositions for parenteral administration that comprise a solution of NAP or ADNF polypeptide, as described above, dissolved or suspended in an acceptable carrier, preferably an aqueous carrier. A variety of aqueous carriers may be used including, for example, water, buffered water, 0.4% saline, 0.3% glycine, hyaluronic acid and the like. These compositions may be sterilized by conventional, well known sterilization techniques or, they may be sterile filtered. The resulting aqueous solutions may be packaged for use as is or lyophilized, the lyophilized preparation being combined with a sterile solution prior to administration. The compositions may contain pharmaceutically acceptable auxiliary substances as required to approximate physiological conditions including pH adjusting and buffering agents, tonicity adjusting agents, wetting agents and the like, such as, for example, sodium acetate, sodium lactate, sodium chloride potassium chloride, calcium chloride, sorbitan monolaurate, triethanolamine oleate, etc.

For solid compositions, conventional nontoxic solid carriers may be used that include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient and more preferably at a concentration of 25%-75%.

For aerosol administration, the NAP or ADNF polypeptides are preferably supplied in finely divided form along with a surfactant and propellant. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. A carrier can also be included, as desired, as with, e.g., lecithin for intranasal delivery. An example includes a solution in which each milliliter included 7.5 mg NaCl, 1.7 mg citric acid monohydrate, 3 mg disodium phosphate dihydrate and 0.2 mg benzalkonium chloride solution (50%) (Gozes et al., J Mol Neurosci. 19(1-2):167-70 (2002)).

In therapeutic applications, the combination of NAP or ADNF polypeptides and other appropriate therapeutics are administered to a patient in an amount sufficient to reduce or eliminate symptoms of Alzheimer's disease, MS, or schizophrenia. An amount adequate to accomplish this is defined as “therapeutically effective dose.” Amounts effective for this use will depend on, for example, the particular NAP or ADNF polypeptide employed, the type of disease or disorder to be prevented, the manner of administration, the weight and general state of health of the patient, and the judgment of the prescribing physician.

For example, an amount of polypeptide falling within the range of a 100 ng to 10 mg dose given intranasally once a day (e.g., in the evening) would be a therapeutically effective amount. Alternatively, dosages may be outside of this range, or on a different schedule. For example, dosages may range from 0.0001 mg/kg to 10,000 mg/kg, and will preferably be about 0.001 mg/kg, 0.1 mg/kg, 1 mg/kg, 5 mg/kg, 50 mg/kg or 500 mg/kg per dose. Doses may be administered hourly, every 4, 6 or 12 hours, with meals, daily, every 2, 3, 4, 5, 6, or 7 days, weekly, every 2, 3, 4 weeks, monthly or every 2, 3 or 4 months, or any combination thereof. The duration of dosing may be single (acute) dosing, or over the course of days, weeks, months, or years, depending on the condition to be treated. Those skilled in the art can determine the suitable dosage, and may rely on preliminary data reported in Gozes et al., 2000, Gozes et al., 2002), Bassan et al. 1999; Zemlyak et al., Regul. Pept. 96:39-43 (2000); Brenneman et al., Biochem. Soc. Trans. 28: 452-455 (2000); Erratum Biochem Soc. Trans. 28:983; Wilkemeyer et al., Proc. Natl. Acad. Sci. USA 100:8543-8548 (2003); review Gozes et al., CNS Drug Rev, 11(4): 353-368 (2005); review Gozes, Pharmacol Ther. 114:146-154 (2007); and review Gozes et al., Curr Alzheimer Res. 6(5):455-460 (2009)).

It must be noted that as used herein and in the appended claims, the singular forms “a,” “and,” and “the” include plural referents unless the context clearly dictates otherwise. Thus, for example, reference to “a nucleic acid” includes a plurality of such nucleic acids and reference to “the polypeptide” includes reference to one or more polypeptides and equivalents thereof known to those skilled in the art, and so forth.

### EXAMPLES

### Example 1

### Combination Treatment of Schizophrenia

### Cell Culture Experiments

Clozapine, a widely used anti psychotic drug may be associated with increased cell mortality. It was not known, however, whether NAP (NAPVSIPQ, SEQ ID NO:2) treatment would inhibit clozapine associated neuronal death. To address this question and validate the efficiency of using a NAP-clozapine combination drug candidate, an experimental test system in cell culture was established (Heiser et al., Journal of Psychopharmacology 21(8):851-856 (2007)). Human SH-SY5Y neuroblastoma supplemented with 15% fetal calf serum (FCS) (Beit-Haemek, Israel), 1% penicillin-streptomycin, and 1% glutamine were incubated in a 5% CO₂ atmosphere. Thirty-six thousand cells/well (in a 96 well plates) were plated 24 hours prior to the clozapine or NAP treatment. Clozapine (CLZ) was obtained from Sigma Chemicals (Sigma, Israel). Thirty-six thousand cells per well (in at least 6 replicate wells) were exposed to clozapine (CLZ, 20 μg/ml) or clozapine (CLZ, 20 μg/ml)+NAP or NAP alone (10⁻¹⁵M-10⁻¹⁰ M) for 24 hours at 37° C. Drug and control solutions were dissolved in ethanol. Measurement of metabolic activity and survival was determined by non-radioactive cell proliferation assay (MTS) (Promega, USA) and evaluated by an ELISA-reader at 490 nm wavelength.

Surprisingly, clozapine induced cell death was reversed by NAP treatment (FIG. 1), although NAP alone did not affect cell mortality (FIG. 2).

Other published research results suggest that clozapine (CLZ) induces tau hyperphosphorylation (Gong et al., Brain Res. 1996 Nov. 25; 741(1-2):95-102). Further studies are designed to assess whether this occurs in this experimental system, in order to provide a possible mechanism for NAP-clozapine combination therapy, as NAP has been shown before to inhibit tau hyperphosphorylation in vivo (Matsuoka et al., J Pharmacol Exp Ther 325, 146-153, 2008; Shiryaev et al., Neurobiol Dis 34, 381-388, 2009; Vulih-Shultzman et al., J Pharmacol Exp Ther 323, 438-449, 2007).

### In Vivo Experiments

### NAP (Davunetide) Enhances Cognitive Behavior in the STOP Heterozygous Mouse—a Microtubule-Deficient Model of Schizophrenia

The study was set out to investigate whether a mouse model of schizophrenia that is associated with cytoskeletal deficits exhibited cognitive deficits and whether chronic intranasal NAP treatment was effective in cognitive enhancement in this model. The stable tubule-only polypeptide (STOP) knockout mice have been shown before to provide a reliable model for schizophrenia. Here, heterozygous STOP mice (STOP+/−) showed schizophrenia-like symptoms (e.g., hyperactivity in open field) that were ameliorated by chronic treatment with clozapine (a clinically used anti-psychotic drug). Following model validation, STOP+/−mice were subjected to daily nasal NAP or vehicle application and compared to similarly treated STOP+/+ mice. NAP treatment significantly decreased open field locomotor activity in the STOP+/− mice. Importantly, STOP+/− mice were significantly impaired in object recognition and were significantly improved to STOP+/+performance level upon NAP treatment. Finally, spatial memory was also impaired in the STOP+/− mice and was ameliorated by NAP treatment. These studies provide support for clinical use of NAP (generic name, davunetide; intranasal formulation: AL-108) on cognitive functions in schizophrenic patients. Further details of this study are provided in Example 3.

An In Vivo Experiment Conducted with Clozapine Alone and with Clozapine+NAP

STOP+/− male mice (6 months old in average, n=4) received daily doses of 10 mg/kg intraperitoneal injection (IP) of clozapine (5 days a week), for the duration of the experiment (5 weeks). Clozapine (10 mg/kg) was solubilized under acidic conditions (pH ˜2.0 with HCl), and the solutions titrated back to ˜pH 7.4 with NaOH. In addition, these mice received daily intranasal vehicle solution that included the following ingredients (per milliliter): 7.5 mg of NaCl, 1.7 mg of citric acid monohydrate, 3 mg of disodium phosphate dihydrate, and 0.2 mg of benzalkonium chloride solution (50%) (Alcalay et al., Neurosci Lett 361, 128-131, 2004).

A combination of clozapine with NAP experiment included an additional four mice (6 months old in average) that were injected with clozapine and administered with NAP intranasally in the vehicle delineated above (Alcalay et al., Neurosci Lett 361, 128-131, 2004). NAP or vehicle solution was administered daily to mice hand-held in a semi-supine position with nostrils facing the investigator. A pipette tip was used to administer 2.5 μl/nostril. The mouse was hand-held until the solution was totally absorbed (˜10 s).

The experiment continued for 5 weeks.

### Results

There was a very high mortality rate in this experiment, precluding the possibility of assessing behavioral outcome. The clozapine group showed 50% death after 2.5 weeks of treatment and an additional death at ˜4.5 weeks (amounting to 75% death). The NAP+clozapine group of mice were relatively spared with only one death occurring ˜4.5 weeks after the initiation of treatment (i.e., 75% survival). The surprising mortality observed is believed to be associated with clozapine-related cell death that is seen in the in vitro cell culture experiments.

Further experiments will be conducted with clozapine at lower doses (at about 1/10 level) to accommodate behavioral assessments. Additional experiments will also be conducted in a pharmacological model that was utilized before (Malkoff et al., J Neural Transm 115, 1563-1571, 2008).

### Example 2

### Combination Treatment of Alzheimer's Disease

A synergistic effect between NAP and Galantamine (AchEI) was studied in a serum-deprivation model. The survival-promoting effects of galantamine (an acetylcholine-esterase inhibitor, or AcEI), NAP, and their combinatorial treatment were assessed in cell cultures. Cell culture method was adapted from Calderon et al., J Neurosci Res 56, 620-631 (1999).

### Material and Methods

### Cell Lines

Rat pheochromocytoma cells (PC12) were grown in Dulbecco's modified Eagle's medium (DMEM) supplemented with 8% fetal calf serum, 8% horse donor serum, 2 mm L-glutamine, and 1% penicillin-streptomycin solution.

For the experiments, cells were harvested, re-suspended, and seeded in 96-well plates at a concentration of 1.5×10⁴ cells/well. Cells were pretreated with NAP and Galantamine [Galantamine (0.1, 0.5, 1, 10 μM); NAP (10⁻⁸M, 10⁻¹° M, 10⁻¹²M, 10⁻¹⁵M)] 3 hours prior to serum deprivation and during serum deprivation. The assay for cell viability was performed 48 hours after serum deprivation. Calibration method is described below.

NAP (Allon Therapeutics Inc.) was dissolved in water at a concentration of 10⁻³M (aliquots were stored in at −20° C.). It was further diluted in medium and finally diluted 1:100 in medium to obtain the required concentrations.

Galantamine, purchased from Sigma, was prepared in a stock solution of 20 mg/ml in PBS and stored in aliquots at −20° C. For the experiments it was diluted 1:100 (to obtain final concentrations) in medium containing NAP.

### Serum Deprivation

Culture medium was replaced by fresh medium without serum, containing NAP and Galantamine. Cells were incubated for 48 hours and then subject to MTS viability assay.

### Treatment Groups

The following groups were tested:

(1) Cells with serum;

(2) Cells without serum;

(3) Cells without serum treated with Galantamine alone;

(4) Cells without serum treated with NAP alone

(5) Cells without serum treated with Galantamine and NAP

### Calibration of the Serum Deprivation Method

PC12 cells were seeded and treated as mentioned above without any drugs for 24 hours, 48 hours, and 72 hours. MTS results are represented by the percentage of viable cells from the serum deprived treatment vs. control treatment.

### Example 3

### NAP Treatment of STOP Heterozygous Mouse

NAP is an 8 amino-acid active peptide (NAPVSIPQ; SEQ ID NO:2) fragment of activity-dependent neuroprotective protein (ADNP), which participates in neurodevelopment and neuroprotection. In mice, ADNP knock-outs are lethal exhibiting CNS dysgenesis, while heterozygous ADNP mice are viable but demonstrate cognitive dysfunction coupled with microtubule-associated protein (tau) pathology. NAP treatment ameliorates, in part, ADNP-associated dysfunctions in vivo. This study was set out to investigate whether a mouse model of schizophrenia that is associated with cytoskeletal deficits exhibited cognitive deficits and whether chronic intranasal NAP treatment was effective in cognitive enhancement in this model. The stable tubule-only polypeptide (STOP) knockout mice have been shown before to provide a reliable model for schizophrenia. Here, heterozygous STOP mice (STOP+/−) showed schizophrenia-like symptoms (hyperactivity in open field and cognitive dysfunction, in a test of object recognition/discrimination) that were ameliorated by chronic treatment with clozapine (a clinically used anti-psychotic drug). Following model validation, STOP+/− mice were subjected to daily nasal NAP or vehicle application and compared to similarly treated STOP+/+ mice. NAP treatment significantly decreased open field locomotor activity in the STOP+/− mice. Importantly, STOP+/− mice were significantly impaired in object recognition and were significantly improved to STOP+/+performance level upon NAP treatment. Finally, spatial memory was also impaired in the STOP+/− mice and was ameliorated by NAP treatment. These studies provide support for further clinical testing of NAP (generic name, davunetide, intranasal formulation—AL-108) on cognitive functions in schizophrenic patients.

### Introduction

AL-108 is a drug candidate that has demonstrated positive effects on cognition in a Phase II study carried out in subjects with amnestic mild cognitive impairment. AL-108 is the intranasal formulation of NAP, an 8 amino-acid peptide (NAPVSIPQ, SEQ ID NO:2, MW=824.9; generic name: davunetide) fragment of activity-dependent neuroprotective protein (ADNP, ˜124 KD), which participates in neurodevelopment and neuroprotection (Bassan M et al., J Neurochem, 72(3): 1283-1293 (1999); Gozes I, Pharmacol Ther, 114(2): 146-154 (2007); Gozes I et al., CNS Drug Rev, 11(4): 353-368 (2005); Mandel S, Gozes I, J Biol Chem, 282(47): 34448-34456 (2007); Mandel S, Rechavi G, Gozes I, Dev Biol, 303(2): 814-824 (2007); Steingart R A, Gozes I, Mol Cell Endocrinol, 252(1-2): 148-153 (2006)). In mice, ADNP knock-outs are lethal and exhibit CNS dysgenesis (Pinhasov A et al., Brain Res Dev Brain Res, 144(1): 83-90 (2003)). While exerting multiple gene regulation effects through chromatin interactions (Mandel S, Gozes I, J Biol Chem, 282(47): 34448-34456 (2007); Mandel S, Rechavi G, Gozes I, Dev Biol, 303(2): 814-824 (2007)), ADNP may mediate some effects through interaction with microtubules (Furman S et al., Neuron Glia Biol, 1(3): 193-199 (2004); Mandel S, Spivak-Pohis I, Gozes I, J Mol Neurosci. (2008)). Because of its large size, it is assumed that ADNP does not penetrate the blood brain barrier and thus cannot be used pharmacologically. NAP, however, is absorbed following intranasal administration, and has been shown to cross the blood brain barrier (Gozes I et al., J Pharmacol Exp Ther, 293(3): 1091-1098 (2000); Gozes I et al., CNS Drug Rev, 11(4): 353-368 (2005)). NAP is a highly active neuroprotectant and is thought to act through microtubule interaction, inhibition of tau hyperphosphorylation and apoptosis (Divinski I et al., J Neurochem, 98(3): 973-984 (2006); Divinski I, Mittelman L, Gozes I, J Biol Chem, 279(27): 28531-28538 (2004); Gozes I, Divinski I, J Alzheimers Dis, 6(6 Suppl): S37-41 (2004), Gozes I, Divinski I, Curr Alzheimer Res, 4(5): 507-509 (2007); Holtser-Cochav M, Divinski I, Gozes I, J Mol Neurosci, 28(3): 303-307 (2006); Leker R R et al., Stroke, 33(4): 1085-1092 (2002); Matsuoka Y et al., J Mol Neurosci, 31(2): 165-170 (2007); Matsuoka Y et al., J Pharmacol Exp Ther, 325(1): 146-153 (2008); Vulih-Shultzman I et al., J Pharmacol Exp Ther, 323(2): 438-449 (2007)).

Microtubules, key elements of the neuronal cytoskeleton (Gozes I, Littauer U Z, Nature, 276(5686): 411-413 (1978)), consist of the major subunits, tubulin and microtubule associate proteins (MAPs) such as tau. Hyperphosphorylation of tau leads to the formation of neurofibrillary tangles, that are formed when tau dissociates from microtubules and clusters into an insoluble mass (Alonso A C et al., Curr Alzheimer Res, 5(4): 375-384 (2008); Avila J et al., J Biomed Biotechnol, 2006(3): 74539 (2006); Trojanowski J Q, Lee V M, Med Clin North Am, 86(3): 615-627 (2002)).

Although neurofibrillary tangles are most associated with cognitive dysfunction in Alzheimer's disease (AD), some increase in neurofibrillary pathology has also been reported in schizophrenia, potentially as a consequence of antipsychotic medication (Eastwood S L et al., J Psychopharmacol, 21(6): 635-644 (2007)). NAP increases the ratio of non-phosphorylated tau to phosphorylated tau, thus, affecting tubulin polymerization into microtubules, resulting in the maintenance of the microtubular network, which is essential for the survival of the cell (Gozes et al, 2004). In vivo, NAP reduces tau hyperphosphorylation and enhances cognitive functions in ADNP-deficient mice (Vulih-Shultzman I et al., J Pharmacol Exp Ther, 323(2): 438-449 (2007)), in a triple transgenic mouse model of AD exhibiting amyloid overload and tau pathology (Matsuoka Y et al., J Mol Neurosci, 31(2): 165-170 (2007); Matsuoka Y et al., J Pharmacol Exp Ther, 325(1): 146-153 (2008)) and in a model of frontotemporal dementia (tauopathy) (Shiryaev et al., Neurobiol Dis. 2009 May; 34(2):381-8. Epub 2009 Mar. 2).

In the brain, tubulin frameworks are stabilized by stable tubule-only polypeptide (STOP) proteins (Bosc C, Andrieux A, Job D, Biochemistry, 42(42): 12125-12132 (2003)) (aka MAP6), a family of MAPs important for microtubule stabilization. Linkages to allelic variation in STOP genes has been reported in schizophrenia, along with altered STOP protein expression in some brain regions (Shimizu H et al., Schizophr Res, 84(2-3): 244-252 (2006)). STOP−/− mice, exhibit synaptic deficits (Andrieux A, et al., Genes Dev, 16(18): 2350-2364 (2002)), disturbances in dopaminergic neurotransmission (Brun P et al., J Neurochem, 94(1): 63-73 (2005)) along with deficits in behavior and hypermotility that are partially reversed with clozapine (Fradley R L et al., Behav Brain Res, 163(2): 257-264 (2005)). Importantly, a paclitaxel-like microtubule stabilizer (epothilone D) ameliorates synaptic function and behavior in this schizophrenia model (Andrieux A, et al., Biol Psychiatry, 60(11): 1224-1230 (2006)). Thus, the neuropathological features of schizophrenia may be, in part, due to abnormal STOPrelated instability of the microtubular structure. These characteristics suggest that NAP treatment may reverse STOP-related deficiencies that produce abnormal neurophysiological processes in schizophrenia.

As the breeding of the STOP-null mice may require special handling due to abnormal nurturing behavior of STOP−/− mothers (Andrieux A, et al., Genes Dev, 16(18): 2350-2364 (2002)), resulting in paucity of progeny, while out-breeding may result in prolific progeny of STOP+/− mice, the STOP+/− mice may offer a new model of potential schizophrenia-like behavioral deficits. To this point, STOP+/− mice were previously shown to express reduced synaptophysin, VGlut 1, GAP-43 and spinophilin mRNAs in the hippocampus, suggesting cognitive impairment (Eastwood S L et al., J Psychopharmacol, 21(6): 635-644 (2007)),

As part of an ongoing translational research program for the intranasal formulation of NAP (davunetide intranasal; AL-108) in cognitive-impairment in schizophrenic patients, it was evaluated here (1) whether partial STOP-deficiency (STOP+/− mice) results in behavioral and cognitive impairments that will allow a simple test for potential future drugs; (2) whether STOP+/− mice treated chronically with clozapine, a clinically relevant antipsychotic, will reduce the positive symptoms (hyper-locomotion) in this model; and (3) whether intranasal NAP administration to STOP+/− mice results in increased cognitive function.

### Materials and Methods

### Animal Model

Founder mice for the production of STOP-null (STOP−/−) and STOP heterozygous (STOP+/−) mouse colonies were obtained under from Annie Andrieux and Didier Job (INSERM). These mice were originally generated on a 50:50 BALBc/129 SvPas with gene targeting being used to replace exon 1 of the STOP gene with a non-functional construct (Andrieux A, et al., Genes Dev, 16(18): 2350-2364 (2002)). As the mRNAs of all of the STOP proteins characterized to date contain this exon (Denarier E et al., Biochem Biophys Res Commun, 243(3): 791-796 (1998a); Denarier E et al., Proc Natl Acad Sci USA 95(11): 6055-6060 (1998b)), the expression of all STOP isoforms is suppressed in the null mice, and is partially suppressed in the heterozygous mice (Eastwood S L et al., J Psychopharmacol, 21(6): 635-644 (2007)).

STOP null mice were mated with BALBc mice and the colony was maintained by continuous cross breeding and DNA profiling for STOP+/− or STOP homozygous (STOP+/+) genotype, as previously described (Andrieux A, et al., Genes Dev, 16(18): 2350-2364 (2002)). As the breeding of the STOP-null mice resulted in paucity of progeny, while out-breeding resulted in prolific progeny of STOP+/− mice, the STOP+/− mice were tested for potential schizophrenia-like behavioral deficits.

All experimental procedures were approved by the Animal Care Committee of the Tel-Aviv University and the Israel Government.

### Experimental Design and Drug Application

Experiments assessed STOP+/− male mouse behavior in comparison to STOP+/+ mice as well as clozapine or NAP efficacy in vivo.

For the initial comparisons of STOP+/− mice to STOP+/+ mice (littermates), each experimental group included 10-12 mice, 4-12 month-old; mean age 6 months, there were no highly significant differences between the tested ages in terms of the behavioral outcome). The STOP+/+ and STOP+/− male mice received daily (5 days a week) NAP nasal application vehicle as follows below for the duration of 7-10 weeks.

The NAP vehicle solution (termed DD), included the following ingredients (per milliliter): 7.5 mg of NaCl, 1.7 mg of citric acid monohydrate, 3 mg of disodium phosphate dihydrate, and 0.2 mg of benzalkonium chloride solution (50%) (Alcalay R N et al., Neurosci Lett, 361(1-3): 128-131 (2004)). NAP or vehicle solution (DD) was administered to mice hand-held in a semisupine position with nostrils facing the investigator. A pipette tip was used to administer 2.5 μl/nostril. The mouse was hand-held until the solution was totally absorbed (10 s). For clozapine experiments, clozapine (10 mg/kg) was solubilized under acidic conditions (pH ˜2.0 with HCl), and the solutions titrated back to ˜pH 7.4 with NaOH. STOP+/− male mice (5 months old, n=4) received daily doses of 10 mg/kg intraperitoneal injection (IP) of clozapine (5 days a week), for the duration of the experiment (7 weeks). A control STOP+/− male mice group (5 month old, n=3) were injected daily with saline. NAP treatment included daily (5 days a week) intranasal administrations (0.5 μg/5 μl/mouse/day) for 7-10 weeks. For intranasal administration, the peptide was dissolved in the vehicle solution (termed DD) described above. Each experimental group included 10-12 mice, 4-12 month-old; mean age 6 months).

Behavioral assessments were implemented according to the following timeline: on the first day of the third or fourth week of drug application, the open field test was conducted; on the first day of the fourth or the sixth week of drug application, the Morris water maze was carried out for 5 days including a probe test on the fifth day of testing; on third day of the fifth or tenth week of drug application, an object recognition test was conducted. All behavioral tests were performed 1 hour after the daily vehicle or drug administration.

### Behavioral Measurements

### Locomotor Activity

The test is based on previous studies which showed that schizophrenic-like behavior in rodents is characterized by hyper-locomotor activity (Andrieux A, et al., Genes Dev, 16(18): 2350-2364 (2002)). Locomotor activity was measured in 5 consecutive sessions, 3 minutes each and the mean path length in cm of each mouse in the 3 minute sessions per the entire 15 minute period of observation in open field (80 cm diameter) was determined. Tracking was performed using the HVS IMAGE-computerized system (HVS Image, Buckinghamshire, U.K.).

Object recognition (Powell K J et al., Behav Neurosci, 121(5): 826-835 (2007))

The test is based on visual discrimination between two different objects in an arena of 30 cm×40 cm. This test contained 2 consecutive days of habituation (five minutes per day) and an experimental day which consisted of two sessions. One hour after the daily intranasal NAP treatment, or clozapine injection, each male mouse was exposed to two identical objects for 5 minutes and the time spent sniffing/touching each object was measured (first daily session). Three hours later, the mice were exposed to one familiar and one novel object for 3 minutes and the time spent sniffing/touching each object was measured again (second session, measuring visual memory). The data was analyzed using two different methods. The first method measured the time spent exploring an object, novel or familiar, and compared the two. The second method evaluated the discrimination capacity of the mice between the novel object and the familiar object using the following formula. When a=time of exploration of the familiar object, and b=time of exploration of the novel object, D1=b−a, E2=a+b, D2, the discrimination capacity, is equal to D1/E2 (de Bruin N, Pouzet B, Pharmacol Biochem Behav, 85(1): 253-260 (2006)).

### Morris Water Maze

The test measures the ability of rodents to learn and remember, by spatial navigation, the place of the hidden platform in a round water maze. This test has been used before to assess NAP function (Gozes I et al., CNS Drug Rev, 11(4): 353-368 (2005)). Here, mice were monitored on five consecutive swimming days. Two consecutive tests were performed daily, giving the mice a 20-s pause on the hidden platform before and between tests. The hidden platform diameter was 15 cm, and pool diameter was 140 cm. Maximal latency time to find the hidden platform was set at 90s. Platform placement was changed daily. The time required to reach the platform during the first daily test (indicative of learning and intact reference memory) and the time required to reach the platform in the second trial [indicative of short-term (working) memory] were measured and recorded separately. On the fifth day, a probe test was performed after the second daily trial. The platform was removed from the maze and the time spent by the mice in the pool's quadrant where the platform used to be was recorded. To control for the animals motivational factors, visual abilities, and motor functions, the animals were tested in the visible platform version of the Morris water maze task. Mice were released into the water and the time taken to find the platform was recorded (maximum 60 seconds). Animals that did not climb on the platform during this test were excluded from the statistical analysis. Tracking was performed using the HVS IMAGE-computerized system (HVS Image, Buckinghamshire, U.K.).

### Statistical Analysis

Statistical tests used data were compared using two way ANOVA, followed by Tukey's posthoc tests. Additional statistical tests included one way-ANOVA and Student's t-test. The threshold of statistical significance for all tests was 5% or 1%. All results are shown as means±SEM.

### Results

### Model Validity and Locomotor Activity

To test for the validity of the STOP+/− model and the potential of NAP in ameliorating STOP deficiencies, after three-five weeks of vehicle treatment, the male mice (mean age ˜7 months at the time of the experiment) were subjected to the open field test as a measure of hyperactivity. The path traveled by each mouse over 5 consecutive 3 minute periods was measured and means were compared between the groups, as indicators for locomotor activity. Results demonstrated a significant increase in the locomotor activity of the STOP+/− mice as compared to the STOP+/+ mice (FIG. 8A; ##P<0.01) validating the STOP+/−mouse model for hyperactivity.

To further validate the STOP+/− model as responsive to a clinically relevant antipsychotic drug, STOP+/− male mice (5 months old) were subjected to clozapine treatment. As expected, five weeks of clozapine treatment resulted in a significant reduction in the STOP+/−mouse activity in the open field test (FIG. 8B**P<0.01). These results are indicative of the predictive validity of the model. None of the clozapine studies reported here included tests of clozapine in the wild type mice as previous studies clearly demonstrated no effect for clozapine treatment in this line of STOP+/+ mice (Andrieux A, et al., Genes Dev, 16(18): 2350-2364 (2002); Fradley R L et al., Behav Brain Res, 163(2): 257-264 (2005)).

Further experiments showed that after three-five weeks of NAP treatment the treated STOP+/− male mice (˜7 months old at the time of the experiment) exhibited significantly reduced the path length in the open field (FIG. 8A; ***P<0.001; Student's t test). Importantly, NAP treatment resulted in behavior of the STOP+/− mice that was similar to the control STOP+/+ mice (FIG. 8A).

To test the hypothesis and justify posthoc pairwise comparisons, a genotype by treatment interaction was also implemented using a two-way ANOVA followed by a Tukey post hoc test showing significance of P<0.001 for NAP effect, significance of P<0.001 for genotype effect and a non significant NAP X genotype effect P=0.420, indicating an effect of NAP also on the control mice (albeit a non-significant effect).

### Model Validity and Object Recognition

To further test for the validity of the STOP+/− model and the potential of NAP in ameliorating STOP deficiencies, on third day of the fifth or tenth week of vehicle application, the male mice (mean age 7-8 months at the time of the experiment) were subjected to the object recognition test, as a measure of cognitive function. In the first session, when the mice were exposed to two identical objects, these mice spent similar time periods with the two objects (data not shown). Three hours after the first object recognition session, the mice were exposed to a novel object, side by side with the familiar object observed 3 hours before. While the STOP+/+ mice showed a significant preference to the novel object, by spending more time with it, the STOP+/− mice were indifferent to the novel object and were even significantly more interested in the familiar object (FIG. 9A).

As expected, the antipsychotic drug (but not cognitive enhancer) clozapine, only showed a trend toward improvement but did not reach a statistical significance in the object recognition test (FIG. 9B; P=0.1). In contrast, NAP treatment was significantly beneficial to the STOP+/− mice that were not able to discriminate between the familiar and novel objects without the treatment (FIG. 9A; Student's t-test, P<0.01).

A genotype by NAP treatment interaction was also implemented using a two-way ANOVA showing F (1,41)=13.879; P<0.001 for NAP effect, F (1,41)=10.756; P=0.002 for genotype effect and a significant NAP X genotype effect F (1,41)=13.684; P<0.001.

### Model Validity and Morris Water Maze

To further test for the validity of the STOP+/− model and to assess the potential of NAP in ameliorating potential STOP-associated deficiencies in spatial learning, the Morris water maze paradigm was performed. On the 1st day of the 4th or the 6th week of vehicle application, the Morris water maze was carried out for 5 days including a probe test on the fifth day of testing. STOP+/− male mice were compared to STOP+/+ control males (mean age 7-7.5 months).

No significant differences were observed in the daily experiments, in the ability of the mice to find the hidden platform. However, as mentioned above, a probe test was preformed on the fifth day of the Morris water maze experiment. This test measures the time spent by the mice in the pool's quarter where the platform used to be, reflective of memory. Our results showed a significant reduction in the time spent where the platform used to be by the STOP+/− mice as compared to the STOP+/+ mice, indicative of cognitive deficits and validating the STOP+/− model for deficits in spatial memory (FIG. 10A; Student's t-test, ##P<0.01).

As for object recognition (FIG. 9B), in the probe test of the Morris water maze, clozapine treatment, while showing a trend for improvement did not significantly affect cognitive performance in the probe test (t-test; P=0.1)

In contrast, further evaluations revealed that the STOP+/− mice that were treated daily with NAP spent significantly more time in the pool's quarter where the platform used to be (Student's t-test, ***P<0.001) than the STOP+/− mice that were treated with vehicle (DD) (FIG. 10A).

As above, to test the hypothesis and justify posthoc pairwise comparisons, a genotype by treatment interaction was also implemented using a two-way ANOVA showing an F (1,33)=4.802; P=0.036 for NAP effect, F (1,33)=1.512; P=0.228 for genotype effect and a significant NAP X genotype effect F (1,33)=8.028; P=0.008.

### Discussion

This study established the STOP+/−mouse model for the testing of potential microtubule interacting, cognition protective drug candidates and provide a useful complement to STOP −/− mice. In contrast to the STOP−/− mice which may require special handling due to abnormal nurturing behavior of STOP−/− mothers (Andrieux A, et al., Genes Dev, 16(18): 2350-2364 (2002)), resulting in paucity of progeny, here, out-breeding of the STOP+/− mice resulted in prolific progeny that did not require special handling. Hyperactivity in the open field which is associated with schizophrenia-related behavior was increased in STOP+/− mice and was significantly ameliorated by clozapine treatment (a clinically used anti-psychotic drug) which validates the STOP+/− model as responsive (at least in part) to antipsychotic treatment. Similarly, deficits in behavior and hyper-motility exhibited by STOP−/− mice have been shown before to be partially reversed by clozapine treatment (Fradley R L et al., Behav Brain Res, 163(2): 257-264 (2005)).

Here, the STOP+/− mice also exhibited significant deficiencies in the object recognition as well as in the Morris water maze probe tests indicative of impaired memory and a trend of improvement upon clozapine treatment was also observed. The mechanism of action of clozapine is primarily through interaction with the dopaminergic system (Faron-Gorecka A et al., Eur Neuropsychopharmacol, 18(9): 682-691 (2008)) as well as the serotonergic system (Tauscher J. et al., Am J Psychiatry 161:1620-1625 (2004)). It may also increase the activity of the cholinergic system in the prefrontal cortex, thus potentially affecting cognition (Stip E, Chouinard S, Boulay L J, Prog Neuropsychopharmacol Biol Psychiatry, 29(2): 219-232 (2005)). However, lethal clozapine-induced gastrointestinal hypomotility has been observed (Palmer S E et al., J Clin Psychiatry, 69(5): 759-768 (2008)) and there is an increased requirement for drug candidates for the direct treatment of cognitive impairments in schizophrenia (Buchanan R W et al., Schizophr Bull, 33(5): 1120-1130 (2007)).

NAP, like clozapine, significantly decreased open field locomotor activity in the STOP+/− mice, although, a similar effect of NAP (albeit non-significant) was also observed on the NAP-treated STOP+/+ mice. Further results indicated that the significant impairments in object recognition/discrimination observed in the STOP+/− mice were significantly ameliorated by daily NAP treatments. Additionally, as indicated above, spatial memory was also significantly impaired in the STOP+/− mice and was significantly ameliorated by NAP treatment.

Previous data suggested that NAP protected cholinergic function, associated with learning and memory (Bassan M et al., J Neurochem, 72(3): 1283-1293 (1999); Gozes I et al., J Pharmacol Exp Ther, 293(3): 1091-1098 (2000)), which have been shown before to be affected in the STOP−/− mice (Bouvrais-Veret C et al., Neuropharmacology, 52(8): 1691-1700 (2007)) and in schizophrenic patients (Stip E, Chouinard S, Boulay L J, Prog Neuropsychopharmacol Biol Psychiatry, 29(2): 219-232 (2005)).

Additional previous data suggested that NAP treatment also improved cognitive performance in normal rodents as compared to vehicle-treated controls (Alcalay R N et al., Neurosci Lett, 361(1-3): 128-131 (2004); Bassan M et al., J Neurochem, 72(3): 1283-1293 (1999); Gozes I et al., J Pharmacol Exp Ther, 293(3): 1091-1098 (2000); Levy A et al., Regul Pept, 109(1-3): 127-133 (2002)). In the current experiments, control mice treated with NAP did not show improvement in their cognitive performance suggesting that this NAP-cognitive-enhancing effect may be dependent on the strain of mice and the experimental paradigm. Previous studies also suggested that NAP had anxiolytic properties in rodents (Alcalay R N et al., Neurosci Lett, 361(1-3): 128-131 (2004)), and may explain, in part the reduction in locomotor activity (associated with psychotic behavior) in the open field observed in the NAP-treated mice. Anti-anxiety effects of NAP may be relevant to cognitive dysfunction in schizophrenia as many schizophrenic patients suffer from anxiety (Braga R J et al., J Psychiatr Res, 39(4): 409-414 (2005)).

Several studies have reported decreased spine density along with decreased expression of neurite-related proteins in schizophrenia (Eastwood S L et al., J Psychopharmacol, 21(6): 635-644 (2007); Hill J J, Hashimoto T, Lewis D A, Mol Psychiatry, 11(6): 557-566 (2006); Kolluri N et al., Am J Psychiatry, 162(6): 1200-1202 (2005)). Furthermore, abnormal neuronal shape, loss of dendrites and spines, and irregular distribution of neuronal elongations occur in specific brain areas of schizophrenic patients including the hippocampus and the cerebral cortex (Benitez-King G et al., Curr Drug Targets CNS Neurol Disord, 3(6): 515-533 (2004); Ito et al., 2005). The primary in vitro support for the use of NAP as a potential protective agent in schizophrenia is its effectiveness in stimulating neurite outgrowth based upon interaction with tubulin/microtubules (Divinski I et al., J Neurochem, 98(3): 973-984 (2006); Smith-Swintosky V L et al., J Mol Neurosci, 25(3): 225-238 (2005)). Importantly, NAP activity was related to tubulin and tau functions, with NAP-associated decreases in tau hyperphosphorylation and NAP-associated relative increases in soluble, potentially functional tau (Divinski I et al., J Neurochem, 98(3): 973-984 (2006); Divinski I, Mittelman L, Gozes I, J Biol Chem, 279(27): 28531-28538 (2004); Gozes et al., 2004; Matsuoka Y et al., J Mol Neurosci, 31(2): 165-170 (2007); Matsuoka Y et al., J Pharmacol Exp Ther, 325(1): 146-153 (2008); Vulih-Shultzman I et al., J Pharmacol Exp Ther, 323(2): 438-449 (2007)). Further studies have shown effects on NAP on neurite outgrowth and neuronal survival visualized by microtubule-associated protein 2 (MAP2) immunoreactivity in neurites (Smith-Swintosky V L et al., J Mol Neurosci, 25(3): 225-238 (2005); Visochek et al., J Neurosci, 25(32): 7420-7428 (2005); Zemlyak I et al., Peptides, 28(10): 2004-2008 (2007)). Assuming that neuronal deficits associated with microtubule dysfunction underlie cognitive dysfunction in schizophrenia, a NAP treatment approach may be effective.

The current study is the first demonstration of NAP effects in a STOP-deficient model. Over the past several years, it has been shown that resistance of microtubules to the cold is largely due to polymer association with STOP proteins. This resistance of microtubules to the cold has been intriguing because MAP2 and tau are devoid of microtubule cold stabilizing activity (Bosc C, Andrieux A, Job D, Biochemistry, 42(42): 12125-12132 (2003)). STOP suppression in mice has been found to induce synaptic defects including synaptophysin reduction even in STOP+/− mice (Eastwood S L et al., J Psychopharmacol, 21(6): 635-644 (2007)) arguing for a central link between microtubule function and synapse formation. NAP has previously been shown to increase synaptophysin in both rat cortical and hippocampal cultures (Smith-Swintosky V L et al., J Mol Neurosci, 25(3): 225-238 (2005)) and to be associated with synaptic plasticity and function (Pascual M, Guerri C, J Neurochem, 103(2): 557-568 (2007)). NAP showed microtubule rearrangement and stabilization of microtubules in astrocytes, protected against tubulin aggregation resulting from zinc toxicity (Divinski I et al., J Neurochem, 98(3): 973-984 (2006)) and reversed microtubule depolymerization induced by nocodazole (Gozes I, Pharmacol Ther, 114(2): 146-154 (2007)). NAP passively entered astrocytes subjected to cold temperature and this was coupled with apparent microtubule reorganization/stabilization (Divinski I, Mittelman L, Gozes I, J Biol Chem, 279(27): 28531-28538 (2004)). Interestingly, a paclitaxel-like microtubule stabilizer (epothilone D) ameliorated synaptic function and behavior in the STOP−/− schizophrenia model (Andrieux A, et al., Biol Psychiatry, 60(11): 1224-1230 (2006)). Since NAP is also postulated to be neuroprotective through its action on microtubules (Divinski I et al., J Neurochem, 98(3): 973-984 (2006)), these observations suggest a potential mechanistic support for NAP protective activity in the STOP mouse model associated with microtubule deficits.

The clinical effect of NAP (davunetide intranasal, AL-108) on cognition has been evaluated in patients with mild cognitive impairment, a precursor of AD. In that study, significant improvement over placebo-treatment was observed in two measures of memory in the NAP-treated (AL-108, intranasal, twice daily) patients, while positive trends were observed in the primary endpoint and secondary endpoints evaluating additional cognitive domains. Headache and nasopharyngeal events were reported more frequently by AL-108-treated subjects. However, the overall incidence of adverse events was similar between placebo- and NAP-treated groups (see allontherapeutics website). These clinical data position AL-108 (davunetide intranasal) as a drug candidate for cognitive protection Alzheimer's disease and other dementias in addition to cognitive impairment associated with schizophrenia (CIAS).

The protective effect of NAP, as demonstrated by reduced hyperactivity in the open field test and enhanced cognitive function in the Morris water maze and object recognition paradigm in the STOP+/− mice, provides a potential mechanistic link between microtubule dysfunction and the positive effects on cognitive impairment associated with schizophrenia (CIAS). CIAS currently presents an intractable indication and our current findings support AL-108 (davunetide intranasal) as a first in class drug candidate for this prevalent devastating indication.

### Example 4

### NAP/Clozapine Combination Treatment of STOP Heterozygous Mouse

The stable tubule-only polypeptide (STOP) knockout mice have been shown to provide a valid model for schizophrenia. The STOP+/−haplodeficient mice were also shown to model schizophrenia (Merenlender-Wagner et al., Peptides 31 (2010) 1368-1373). A cohort of STOP+/− mice (n=54) was subjected to experiments assessing NAP (davunetide intranasal, 0.5 microgram/mouse/day) and clozapine (injection, 3 mg/kg/day) in five behavioral tests: open field activity (or locomotor activity), object recognition, and Morris water maze as described in Example 3 and also in Merenlender-Wagner et al., Peptides 31 (2010) 1368-1373, elevated plus maze (Alcalay et al., Neurosci Lett 361, 128-131, 2004) and social recognition as further described below.

In the open field test, results showed differences among the control groups (saline; NAP vehicle, termed DD; and the combination of saline and DD). Saline treatment reduced hyperactivity in the open field test and the group treated with saline+DD behaved similar to normal mouse control (STOP+/+), whereas NAP+clozapine combination treatment significantly reduced activity (which is recognized as being associated with the schizophrenia-like conditions). In the object recognition test and the Morris water maze, the controls did not differ and were grouped together. Taken these three behavioral tests together, the results indicate: (1) a potential additive effect for NAP and clozapine in the object recognition test; (2) an inhibitory effect of clozapine in the Morris water maze; and (3) protection against adverse clozapine effects on learning by NAP treatment.

As shown in FIG. 11, open field activity was reduced in STOP+/− mice treated with clozapine. NAP treatment also reduced the activity but NAP+clozapine combination treatment substantially increased the level of activity reduction.

In the object recognition test, the animals' capability to discriminate between a novel object and a familiar object was calculated and indicated in FIG. 12 as discrimination capacity D2. Compared to the pooled control groups (CONT, STOP+/− mice of three control groups saline, DD, saline+DD), each of clozapine and NAP alone was effective to increase memory scores, whereas NAP+clozapine combination treatment showed an additional effect.

In the Morris Water Maze test, the mice were tested for their ability to find the location of a hidden platform in water maze. The time spent searching reflects learning and memory, and the time spent in the area where the platform used to be reflects memory. The second test, which measures the time spent in the area where the platform used to be and is performed after the 5 learning days, is called the Probe Test. As shown in FIG. 13 and FIG. 14, the presence of NAP in NAP+clozapine combination treatment increased memory scores, demonstrating NAP's ability to protect animals from clozapine's negative effect on animals' memory.

The two additional tests performed in this study were the elevated plus maze test and social recognition test. The elevated plus maze test is the most widely used model of anxiety-like behaviors for drug discoveries. The animal is placed on the center of elevated 4-arm maze. Among the four arms two are closed and dark, whereas the other two are open and brightly lit. The behavior of the animal depends on the naturalistic conflict between the tendency of the animal to explore a novel environment and the aversive properties of a brightly lit, open area. 8-month old mice (STOP+/−) were placed into the center of the plus maze for 300 seconds and the number of seconds spent in the open and closed arms was recorded. As shown in FIG. 15, clozapine-treated mice spent significantly less time in the open arms (indicating curiosity) and longer in the closed arms (indicating anxiety), whereas NAP+clozapine treated mice behaved like the controls.

In the social recognition test, 8-month-old mice (STOP+/−) were habituated for 20 minutes in a cage and then exposed to a new juvenile mouse (1.5-month old) for one minute—followed by a 30-minute interval and another 1 minute exposure four times (familiarizing the test subject with the juvenile mouse). The fifth exposure session included a novel juvenile mouse. Time spent interacting with the juvenile mouse was recorded. As shown in FIG. 16, while the clozapine-treated mice showed somewhat erratic behavior, NAP+clozapine treated mice behaved in a similar manner to normal controls.

In summary, NAP (davunetide) has shown activity in a schizophrenia model (STOP+/−) in vivo (Merenlender-Wagner et al., 2010, supra). This study confirms the activity. Davunetide has also shown activity in a human clinical trial in schizophrenia patients (Javitt D C, Schizophrenia Research 117, 118 (2010). The observations described above show that davunetide protects against adverse effects of clozapine in the Morris water maze (inhibition of learning) and potentiates the insignificant clozapine effect in the object recognition test. In the elevated plus test, the clozapine-treated mice showed significant anxiety, which is consistent with the reported clozapine effect on augmentation of anxiety seen in patients (e.g., Pallanti et al., J Clin Psychiatry. 1999 December; 60(12):819-23). The NAP (davunetide)+clozapine treated mice behave like the controls. This study also showed an effect of clozapine on social recognition (interaction), which is partially ameliorated by co-treatment with NAP (davunetide). Given that olanzapine is structurally similar to clozapine but has a different receptor affinity profile (Mcilwain et al., Neuropsychiatr Dis Treat. 2011; 7: 135-149), we believe that NAP (davunetide) will protect also against unwanted side effects associated with clozapine-like drugs including olanzapine, as well as other similar anti-psychotic drugs.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. All citations are incorporated herein by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed is:

1. A method of treating or preventing neurodegeneration caused by a dementia-related disorder in a human subject in need of such treatment, wherein the dementia-related disorder is a tauopathy-related dementia or aging-related dementia, the method comprising the step of administering to the human subject a therapeutically effective amount of
a) an ADNF III polypeptide comprising an active core site having the following amino acid sequence: Asn-Ala-Pro-Val-Ser-Ile-Pro-Gln (SEQ ID NO:2); and
b) an acetylcholinesterase inhibitor.

2. The method of claim 1, wherein the ADNF III polypeptide is a full length ADNF III polypeptide.

3. The method of claim 1, wherein the ADNF III polypeptide has the formula (R¹)ₓ-Asn-Ala-Pro-Val-Ser-Ile-Pro-Gln-(R²)_{y} (SEQ ID NO:13) in which
R¹ is an amino acid sequence comprising from 1 to about 40 amino acids wherein each amino acid is independently selected from the group consisting of naturally occurring amino acids and amino acid analogs;
R² is an amino acid sequence comprising from 1 to about 40 amino acids wherein each amino acid is independently selected from the group consisting of naturally occurring amino acids and amino acid analogs; and
x and y are independently selected and are equal to zero or one.

4. The method of claim 1, wherein the ADNF III polypeptide is Asn-Ala-Pro-Val-Ser-Ile-Pro-Gln (SEQ ID NO:2).

5. The method of claim 1, wherein the active core site of the ADNF III polypeptide comprises at least one D-amino acid.

6. The method of claim 1, wherein the active core site of the ADNF III polypeptide comprises all D-amino acids.

7. The method of claim 1, wherein the ADNF III polypeptide is a member selected from the group consisting of:
(SEQ ID NO: 9)
Gly-Gly-Asn-Ala-Pro-Val-Ser-Ile-Pro-Gln;
(SEQ ID NO: 10)
Leu-Gly-Gly-Asn-Ala-Pro-Val-Ser-Ile-Pro-Gln-Gln-
Ser;
(SEQ ID NO: 11)
Leu-Gly-Leu-Gly-Gly-Asn-Ala-Pro-Val-Ser-Ile-Pro-
Gln-Gln-Ser;
(SEQ ID NO: 12)
Ser-Val-Arg-Leu-Gly-Leu-Gly-Gly-Asn-Ala-Pro-Val-
Ser-Ile-Pro-Gln-Gln-Ser;
and
(SEQ ID NO: 2)
Asn-Ala-Pro-Val-Ser-Ile-Pro-Gln.

8. The method of claim 1, wherein the ADNF III polypeptide comprises up to about 20 amino acids at one or both of the N-terminus and the C-terminus of the active core site.

9. The method of claim 1, wherein ADNF III polypeptide contains a covalently bound lipophilic moiety to enhance penetration or activity.

10. The method of claim 1, wherein the acetylcholinesterase inhibitor is a member selected from the group consisting of huperzine, Huprines, methanesulfonyl fluoridemetrifonate, physostigmine, neostigmine, pyridostigmine, ambenonium, demarcarium, rivastigmine, galantamine, donepezil, Tacrine, Edrophonium, Phenothiazine, 4-Benzyl-2-(A-Naphtyl)-1,2,4-Thiadiazolidine-3,5-Dione, and rasaginile (azilect).

11. The method of claim 1, wherein the tauopathy is Alzheimer's disease, frontotemporal dementia, or progressive supranuclear palsy.

12. A method of treating or preventing a symptom of multiple sclerosis in a human subject in need of such treatment, the method comprising the step of administering to the human subject a therapeutically effective amount of
a) an ADNF III polypeptide comprising an active core site having the following amino acid sequence: Asn-Ala-Pro-Val-Ser-Ile-Pro-Gln (SEQ ID NO:2); and
b) glatiramer acetate.

13. The method of claim 12, wherein the ADNF III polypeptide is a full length ADNF III polypeptide.

14. The method of claim 12, wherein the ADNF III polypeptide has the formula (R¹)ₓ-Asn-Ala-Pro-Val-Ser-Ile-Pro-Gln-(R²)_{y} (SEQ ID NO:13) in which
R¹ is an amino acid sequence comprising from 1 to about 40 amino acids wherein each amino acid is independently selected from the group consisting of naturally occurring amino acids and amino acid analogs;
R² is an amino acid sequence comprising from 1 to about 40 amino acids wherein each amino acid is independently selected from the group consisting of naturally occurring amino acids and amino acid analogs; and
x and y are independently selected and are equal to zero or one.

15. The method of claim 12, wherein the ADNF III polypeptide is Asn-Ala-Pro-Val-Ser-Ile-Pro-Gln (SEQ ID NO:2).

16. The method of claim 12, wherein the active core site of the ADNF III polypeptide comprises at least one D-amino acid.

17. The method of claim 12, wherein the active core site of the ADNF III polypeptide comprises all D-amino acids.

18. The method of claim 12, wherein the ADNF III polypeptide is a member selected from the group consisting of:
(SEQ ID NO: 9)
Gly-Gly-Asn-Ala-Pro-Val-Ser-Ile-Pro-Gln;
(SEQ ID NO: 10)
Leu-Gly-Gly-Asn-Ala-Pro-Val-Ser-Ile-Pro-Gln-Gln-
Ser;
(SEQ ID NO: 11)
Leu-Gly-Leu-Gly-Gly-Asn-Ala-Pro-Val-Ser-Ile-Pro-
Gln-Gln-Ser;
(SEQ ID NO: 12)
Ser-Val-Arg-Leu-Gly-Leu-Gly-Gly-Asn-Ala-Pro-Val-
Ser-Ile-Pro-Gln-Gln-Ser;
and
(SEQ ID NO: 2)
Asn-Ala-Pro-Val-Ser-Ile-Pro-Gln.

19. The method of claim 12, wherein the ADNF III polypeptide comprises up to about 20 amino acids at one or both of the N-terminus and the C-terminus of the active core site.

20. The method of claim 12, wherein ADNF III polypeptide contains a covalently bound lipophilic moiety to enhance penetration or activity.

21. A method of treating or preventing schizophrenia in a human subject in need of such treatment, the method comprising the step of administering to the human subject a therapeutically effective amount of
a) an ADNF III polypeptide comprising an active core site having the following amino acid sequence: Asn-Ala-Pro-Val-Ser-Ile-Pro-Gln (SEQ ID NO:2); and
b) an antipsychotic drug.

22. The method of claim 21, wherein the ADNF III polypeptide is a full length ADNF III polypeptide.

23. The method of claim 21, wherein the ADNF III polypeptide has the formula (R¹)ₓ-Asn-Ala-Pro-Val-Ser-Ile-Pro-Gln-(R²)_{y} (SEQ ID NO:13) in which
R¹ is an amino acid sequence comprising from 1 to about 40 amino acids wherein each amino acid is independently selected from the group consisting of naturally occurring amino acids and amino acid analogs;
R² is an amino acid sequence comprising from 1 to about 40 amino acids wherein each amino acid is independently selected from the group consisting of naturally occurring amino acids and amino acid analogs; and
x and y are independently selected and are equal to zero or one.

24. The method of claim 21, wherein the ADNF III polypeptide is Asn-Ala-Pro-Val-Ser-Ile-Pro-Gln (SEQ ID NO:2).

25. The method of claim 21, wherein the active core site of the ADNF III polypeptide comprises at least one D-amino acid.

26. The method of claim 21, wherein the active core site of the ADNF III polypeptide comprises all D-amino acids.

27. The method of claim 21, wherein the ADNF III polypeptide is a member selected from the group consisting of:
(SEQ ID NO: 9)
Gly-Gly-Asn-Ala-Pro-Val-Ser-Ile-Pro-Gln;
(SEQ ID NO: 10)
Leu-Gly-Gly-Asn-Ala-Pro-Val-Ser-Ile-Pro-Gln-Gln-
Ser;
(SEQ ID NO: 11)
Leu-Gly-Leu-Gly-Gly-Asn-Ala-Pro-Val-Ser-Ile-Pro-
Gln-Gln-Ser;
(SEQ ID NO: 12)
Ser-Val-Arg-Leu-Gly-Leu-Gly-Gly-Asn-Ala-Pro-Val-
Ser-Ile-Pro-Gln-Gln-Ser;
and
(SEQ ID NO: 2)
Asn-Ala-Pro-Val-Ser-Ile-Pro-Gln.

28. The method of claim 21, wherein the ADNF III polypeptide comprises up to about 20 amino acids at one or both of the N-terminus and the C-terminus of the active core site.

29. The method of claim 21, wherein ADNF III polypeptide contains a covalently bound lipophilic moiety to enhance penetration or activity.

30. The method of claim 21, wherein the antipsychotic drug is a member selected from the group consisting of Aripiprazole, Clozapine, Ziprasidone, Resperidone, Quetiapine, and Olanzapine.

31. The method of claim 21, wherein the ADNF III polypeptide is Asn-Ala-Pro-Val-Ser-Ile-Pro-Gln (SEQ ID NO:2) and the antipsychotic drug is Clozapine.

32. The method of claim 21 or 31, wherein the ADNF III polypeptide is administered by intranasal administration or systemic administration and the antipsychotic drug is administered by injection or oral administration.

33. The method of claim 21 or 31, wherein the ADNF III polypeptide and the antipsychotic drug are administered together in one composition.

34. The method of claim 31, wherein the ADNF III polypeptide is administered in a sufficient amount to reduce side effects of Clozapine.


SEQUENCE LISTING (20 sequences)

SEQ ID NO: 1 - protein, 9 aa, Artificial Sequence
  synthetic activity dependent neurotrophic factor I (ADNF I) shortest active form (SAL, ADNF-9), ADNF I core active site
SALLRSIPA

SEQ ID NO: 2 - protein, 8 aa, Artificial Sequence
  synthetic activity dependent neuroprotective protein (ADNP, ADNF III) active core site (NAP), ADNF III polypeptide
NAPVSIPQ

SEQ ID NO: 3 - protein, 14 aa, Artificial Sequence
  synthetic ADNF I polypeptide
VLGGGSALLRSIPA

SEQ ID NO: 4 - protein, 19 aa, Artificial Sequence
  synthetic ADNF I polypeptide
VEEGIVLGGGSALLRSIPA

SEQ ID NO: 5 - protein, 13 aa, Artificial Sequence
  synthetic ADNF I polypeptide
LGGGSALLRSIPA

SEQ ID NO: 6 - protein, 12 aa, Artificial Sequence
  synthetic ADNF I polypeptide
GGGSALLRSIPA

SEQ ID NO: 7 - protein, 11 aa, Artificial Sequence
  synthetic ADNF I polypeptide
GGSALLRSIPA

SEQ ID NO: 8 - protein, 10 aa, Artificial Sequence
  synthetic ADNF I polypeptide
GSALLRSIPA

SEQ ID NO: 9 - protein, 10 aa, Artificial Sequence
  synthetic ADNF III polypeptide
GGNAPVSIPQ

SEQ ID NO: 10 - protein, 13 aa, Artificial Sequence
  synthetic ADNF III polypeptide
LGGNAPVSIPQQS

SEQ ID NO: 11 - protein, 15 aa, Artificial Sequence
  synthetic ADNF III polypeptide
LGLGGNAPVSIPQQS

SEQ ID NO: 12 - protein, 18 aa, Artificial Sequence
  synthetic ADNF III polypeptide
SVRLGLGGNAPVSIPQQS

SEQ ID NO: 13 - protein, 88 aa, Artificial Sequence
  synthetic ADNF III polypeptide, NAP related peptide
XXXXXXXXXXXXXXXXXXXXXXXXXXXXXXXXXXXXXXXXNAPVSIPQXXXXXXXXXXXXXXXXXXXXXXXXXXXXXXXXXXXXXXXX

SEQ ID NO: 14 - protein, 5 aa, Artificial Sequence
  synthetic 1-R or 2-R within the formula for ADNF I polypeptide
VLGGG

SEQ ID NO: 15 - protein, 4 aa, Artificial Sequence
  synthetic 2-R within the formula for ADNF I polypeptide
VLGG

SEQ ID NO: 16 - protein, 5 aa, Artificial Sequence
  synthetic 2-R within the formula for ADNF I polypeptide
VLGGV

SEQ ID NO: 17 - protein, 5 aa, Artificial Sequence
  synthetic 2-R within the formula for ADNF I polypeptide
GVLGG

SEQ ID NO: 18 - protein, 5 aa, Artificial Sequence
  synthetic 1-R within the formula for ADNF I polypeptide
LGLGG

SEQ ID NO: 19 - protein, 8 aa, Artificial Sequence
  synthetic 1-R within the formula for ADNF I polypeptide
SVRLGLGG

SEQ ID NO: 20 - protein, 89 aa, Artificial Sequence
  synthetic ADNF I polypeptide, SAL related peptide
XXXXXXXXXXXXXXXXXXXXXXXXXXXXXXXXXXXXXXXXSALLRSIPAXXXXXXXXXXXXXXXXXXXXXXXXXXXXXXXXXXXXXXXX